(19) United States
(12) Patent Application Publication
(10) Pub. No.: US 2013/0102652 A1
(43) Pub. Date: Apr. 25, 2013

(54) METHODS AND COMPOSITIONS RELATED TO MODIFIED ADENOSINES FOR CONTROLLING OFF-TARGET EFFECTS IN RNA INTERFERENCE

(71) Applicants: Cynthia J. Burrows, Salt Lake City, UT (US); Uday Ghanty, Salt Lake City, UT (US)

(72) Inventors: Cynthia J. Burrows, Salt Lake City, UT (US); Uday Ghanty, Salt Lake City, UT (US)

(73) Assignee: UNIVERSITY OF UTAH RESEARCH FOUNDATION, Salt Lake City, UT (US)

(21) Appl. No.: 13/636,295

(22) PCT Filed: Mar. 23, 2011
(86) PCT No.: PCT/US11/29532

§ 371 Date: Dec. 22, 2012

Related U.S. Application Data
(60) Provisional application No. 61/316,650, filed on Mar. 23, 2010.

(51) Int. Cl. C12N 15/113; C07H 23/00; C07H 19/20

(52) U.S. Cl. CPC C12N 15/1137; C07H 19/20; C07H 23/00
USPC 514/44 A; 536/26.7; 536/24.5

(57) ABSTRACT

Disclosed are compositions and methods related to modified nucleobases. Also disclosed are compositions and methods related to modified interfering RNAs. Also disclosed are compositions and methods related to modified adenonsine for controlling off-target effects in RNA interference.

### CROSS-REFERENCE TO RELATED APPLICATIONS

This application claims priority to U.S. Provisional Application No. 61/316,650 filed on Mar. 23, 2010, which is incorporated herein by reference in its entirety.

### STATEMENT REGARDING FEDERALLY SPONSORED RESEARCH

This invention was made with government support under NIH grant number R01 GM 080784. The government has certain rights in this invention.

### BACKGROUND

The drug discovery process enjoyed a huge boost at the beginning of this century with the Noble Prize-winning discovery of long dsRNA (double-stranded RNA) mediated RNAi (RNA interference) in the worm (Fire et al. 1998) and the subsequent demonstration that RNAi, mediated by small-interfering RNA (siRNA), also operates in mammalian cells (Elbashir et al., 2001). New researches sprouted quickly in order to understand the RNAi mechanism and the possibility of its applications as a drug. It has been proposed that siRNA has several advantages over other available therapeutic agents (Bumcrot et al. 2006). To date at least three different siRNAs for indications such as age-related macular degeneration (AMD), a leading cause of blindness, and for respiratory syncytial virus (RSV), have completed phase I clinical trails (Michels et al. 2006, Barik et al. 2006).

siRNAs can be synthetically prepared dsRNA that can sometime range from 19-23 nucleotide long and are similar to miRNAs (micro RNAs) that are formed from long double-stranded RNA by the action of the proteins drosha and dicer. Together they can form the RISC(RNA interference silencing complex) containing Ago2 (Argonaute 2) and result in the cleavage of the targeted mRNA ultimately knocking down the expression of the desired gene (Rand et al. 2004, Ma et al. 2005, Matranga et al. 2005, Rand et al. 2005, Chiu et al. 2002).

However, when long dsRNA is injected into mammalian cells to knock down a gene, it is mostly recognized as a molecular pattern associated with viral infection. This is because many viruses have dsRNA genomes or use RNA-dependent RNA polymerases, which generate long, dsRNA products. Elbashir et al. reported that 21 bp RNA duplexes mimicking miRNAs can be added to mammalian cells and elicit potent, target-specific gene silencing and this led to the great advancement in the field of siRNA.

Despite many advantages of siRNAs, there are certain issues that need to be solved to make it a potent therapeutic agent. For example, stability of siRNAs in intracellular and extra cellular environments (Zimmermann et al. 2006, Morrissey et al. 2005, Soutschek et al. 2004), sequence independent off target effects such as binding with dsRBM proteins including PKR (RNA dependent protein kinase) and ADAR (Adenosine deaminase) (Sledz et al. 2003, Karikó et al. 2004, Yang et al. 2005), sequence dependent off target effects such as binding with genes other than target gene due to partial complementary of siRNA and other immunostimulatory effects (Hemmi et al. 2000, Judge et al. 2005, Hornung et al. 2005), and cellular permeability (Rand et al. 2005) can all be improved.

For example, soluble duplex RNA-binding proteins are potential sources of off-target effects (Sledz et al. 2006; Yang et al. 2005). Furthermore, RNA binding containing dsRBMs (double stranded RNA-binding motifs) such as PKR can interfere with the desired RNA interference effect of a siRNA duplex. (Puthenveetil et al. 2004). High resolution structures solved both by NMR and by X-ray crystallography show these motifs bind ˜16 bp of dsRNA by making contacts in two consecutive minor grooves and the opening to the intervening major groove (Ryter et al. 1998, Blaszczyk et al. 2004, Wu et al. 2004). One study showed many of the cellular proteins capable of binding a biotinylated siRNA duplex contained mainly dsRBMs, including the RNA-dependent protein kinase (PKR) (Zhang et al. 2005). Since dsRBMs bind duplex RNA by making contacts in the minor groove, they introduced a steric block at specific sites in the minor groove and analyzed the effect on PKR binding by affinity cleavage experiments (Vuyisich et al. 2002).

Targeted silencing of disease-associated genes by chemically modified siRNA holds considerable promise as a novel therapeutic strategy. However, unmodified siRNA can exhibit off-target effects.

Disclosed herein are compositions and methods for overcoming these limitations. For example, disclosed herein are compositions and methods comprising modifications of siRNA that result in a reduction or complete abrogation of these off-target effects.

### SUMMARY

Disclosed are compositions comprising modified nucleobases, as well as methods of synthesizing and using such compositions. Also disclosed are compositions that relate to methods of blocking binding of an off-target molecule to an siRNA molecule. Also disclosed are compositions and methods comprising modifying at least one adenosine of the siRNA molecule.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic of base-switching to place a steric blockade in the minor groove during delivery and in the major groove of siRNA during base pairing with mRNA in the RISC;

FIG. 2A shows modifications to the sense and antisense strands at switchable and non-switchable positions; FIG. 2B shows a schematic for switchable and persistent steric crowding in the minor groove using 8-methoxyadenosine modifications in the anti-sense strand of siRNA; FIG. 2C shows a schematic of BndG modifications at positions 6, 9, 11, and 14 of the sense strand to effect disruption of the PKR interactions;

FIG. 3 shows a schematic for the synthesis of a modified adenosine phosphoramidite, which can be incorporated into the antisense strand of dsRNA;

FIG. 4A shows the synthesis of 3′,5′-O-di-t-butylsilyl-2′-O-t-butyldimethyldimethylsilyl-8-propargyloxyadenosine, FIG. 4B shows the synthesis of N⁶-Benzoyl-3′,5′-O-di-t-butylsilyl-2′-O-t-butyldimethyldimethylsilyl-8-propargyloxyadenosine; FIG. 4C shows the synthesis of N⁶-Benzoyl-2′-O-t-butyldimethylsilyl-8-propargyloxyadenosine; FIG. 4D shows the synthesis of 5′-O-(4,4′-Dimethoxytrityl)-N⁶-benzoyl-2′-O-tbutyldimethylsilyl-8-propargyloxyadenosine; and FIG. 4E shows the synthesis of 5′-O-(4,4′-Dimethoxytrityl)-2′-O-(t-butyldimethylsilyl)-N⁶-Benzoyl)-8-propargyloxyadenosine 3′-N,N-diisopropyl(cyanoethyl)-phosphoramidite;

FIG. 5A shows a schematic for the design of the recombinant psiCheck-2 vector including the sequence of the sense and anti-sense strands (shown in FIG. 5B);

FIG. 6 shows a schematic for sequencing the recombinant plasmid;

FIG. 7A shows a positive control siRNA and negative control siRNA; FIG. 7B shows a schematic for an siRNA with a single modification, such as 8-proparglyoxyadenosine, 8-phenethyloxyadenosine, and 8-cyclohexylethyloxyadenosine, at position 4 (4AS), position 6 (6AS), position 10 (10AS), and position 15 (15AS); and FIG. 7C shows the knock-down of caspase 2 by positive control siRNAs at a concentration of 50 nM, 10 nM, and 1 nM;

FIGS. 8A and 8B show the knock down of caspase 2 by propargyl-modified siRNAs at a concentration of 50 nM and 100 nM (Prg4 is a propargyl modification at position 4, Prg6 is a propargyl modification at position 6, Prg10 is a propargyl modification at position 10, Prg15 is a propargyl modification at position 15 as seen in FIG. 7B);

FIG. 9A shows a schematic for an siRNA with modifications, such as 8-propargyloxyadenosine, 8-phenethyloxyadenosine, and 8-cyclohyexylethyloxyadenosine, at multiple positions, specifically at positions 4, 9, 13, and 16; FIG. 9B shows a schematic for an siRNA with modifications at multiple positions, specifically at positions 6 and 10 (6,10-AS), at positions 6 and 15 (6,15-AS), at positions 10 and 15 (10,15-AS), and at positions 6, 10, and 15 (6,10,15-AS);

FIG. 10 shows a schematic for click chemistry;

FIG. 11 shows a schematic for using T_{M} analysis to analyze base switching and click chemistry;

FIG. 12 shows the knock down of caspase 2 by singly modified siRNAs at a concentration of 50 nM, 10 nM, and 1 nM; and

FIG. 13 shows the knock down of caspase 2 by multiply modified siRNAs at a concentration of 50 nM, 10 nM, and 1 nM.

### DETAILED DESCRIPTION

Before the present compounds, compositions, articles, devices, and methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and in the claims that follow, reference will be made to a number of terms which shall be defined to have the following meanings:

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

The term “interfering RNA” or “RNAi” or “interfering RNA sequence” refers to double-stranded RNA (i.e., duplex RNA) that is capable of reducing or inhibiting expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA may have substantial or complete identity to the target gene or may comprise a region of mismatch (i.e., a mismatch motif). The sequence of the interfering RNA can correspond to the full length target gene, or a subsequence thereof.

Interfering RNA includes “short interfering RNA,” “siRNA,” “short interfering nucleic acid,” “antisense RNA” or “siRNA,” e.g., interfering RNA of about 15-60, 15-50, or 15-40 (duplex) nucleotides in length, more typically about, 15-30, 15-25 or 19-25 (duplex) nucleotides in length, and is preferably about 20-24, 21-22, or 21-23 (duplex) nucleotides in length (e.g., each complementary sequence of the double stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecules assembled from two separate oligonucleotides, wherein one strand is the sense strand and the other is the complementary antisense strand; a double-stranded polynucleotide molecule assembled from a single oligonucleotide, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

“Modified interfering RNA” refers to interfering RNA that comprises at least one modified nucleoside described herein, e.g., modified adenosine. Modified interfering RNA targeting can mediate potent silencing of the target sequence. Modified interfering RNA can reduce or completely abrogate the off-target response to interfering RNA.

“Modified nucleoside”, “modified nucleotide” or “modified base” refers to a nucleoside or nucleotide comprising an alteration, change in chemical structure, or addition to a purine ring. For example, a “modified nucleoside”, “modified nucleotide” or “modified base” can refer to a compound comprising formula (I) or formula (II), as well as the additional embodiments of the formulas, as described herein. A “modified nucleoside”, “modified nucleotide” or “modified base” can refer to a “modified adenosine” or “modified adenosine base” wherein the adenosine comprises formula (I) or formula (II), as well as the additional embodiments of the formulas described herein. The modified nucleosides (e.g., modified adenosine) disclosed herein can also be used with interfering RNA. Interfering RNA can be designed to interact with a target nucleic acid molecule through either canonical or non-canonical base pairing. A target nucleic acid molecule can be any nucleic acid. For example a “target nucleic acid molecule” can be DNA, RNA, cDNA, mRNA, or a DNA/RNA hybrid. A target molecule can be a protein or gene of interest.

A “gene of interest” or “sequence of interest” can include one or more transcriptional regulatory sequences and any other nucleic acid, such as introns, that may be necessary for optimal expression of a selected or target nucleic acid. The term “gene of interest” or “sequence of interest” can mean a nucleic acid sequence (e.g., a therapeutic gene), that is partly or entirely heterologous, i.e., foreign, to a cell into which it is introduced. The term “gene of interest” or “sequence of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). The term “gene of interest” or “sequence of interest” can also mean a nucleic acid sequence that is partly or entirely complementary to an endogenous gene of the cell into which it is introduced.

A “protein of interest” means a peptide or polypeptide sequence (e.g., a therapeutic protein), that is expressed from a sequence of interest or gene of interest.

The interaction of the interfering RNA and the target molecule is designed to promote the destruction of the target molecule through, for example, RNAseH mediated RNA-DNA hybrid degradation. Alternatively the interfering RNA is designed to interrupt a processing function that normally would take place on the target molecule, such as transcription or replication. Interfering RNA can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that interfering RNAs bind the target molecule with a dissociation constant (k_{d}) less than or equal to 10⁻⁶, 10⁻⁸, 10⁻¹⁰, or 10⁻¹². A representative sample of methods and techniques which aid in the design and use of interfering RNAs can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

siRNA can be chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al. 2002; Calegari et al. 2002; Byrom et al. 2003; Kawasaki et al. 2003; Knight and Bass 2001; and Robertson et al. 1968). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “mismatch motif” or “mismatch region” refers to a portion of an siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

An “effective amount” or “therapeutically effective amount” of an siRNA is an amount sufficient to produce the desired effect, e.g., an inhibition of expression of a target sequence in comparison to the normal expression level detected in the absence of the siRNA. Inhibition of expression of a target gene or target sequence is achieved when the value obtained with the siRNA relative to the control is about 90%, 80%, 70%, 60%, 50%, 40%, 30%, 25%, 20%, 15%, 10%, 5%, or 0%. Suitable assays for measuring expression of a target gene or target sequence include, e.g. examination of protein or mRNA levels using techniques known to those of skill in the art such as dot blots, northern blots, in situ hybridization, ELISA, immunoprecipitation, enzyme function, as well as phenotypic assays known to those of skill in the art.

As used herein, the term “responder cell” refers to a cell, for example a mammalian cell, that produces a detectable response when contacted with an siRNA.

“Substantial identity” refers to a sequence that hybridizes to a reference sequence under stringent hybridization conditions, or to a sequence that has a specified percent identity over a specified region of a reference sequence.

The phrase “stringent hybridization conditions” refers to conditions under which an siRNA will hybridize to its target sequence, typically in a complex mixture of nucleic acids, but to no other sequences. Stringent hybridization conditions are sequence-dependent and will be different in different circumstances. Longer sequences hybridize specifically at higher temperatures. An extensive guide to the hybridization of nucleic acids is found in Tijssen 1993. Generally, stringent hybridization conditions are selected to be about 5-10° C. lower than the thermal melting point for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at T_{M}, 50% of the probes are occupied at equilibrium). Stringent hybridization conditions may also be achieved with the addition of destabilizing agents such as formamide. For selective or specific hybridization, a positive signal is at least two times background, preferably 10 times background hybridization.

Exemplary stringent hybridization conditions can be as follows: 50% formamide, 5×SSC, and 1% SDS, incubating at 42° C., or 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. 1990.

Nucleic acids that do not hybridize to each other under stringent hybridization conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, Ausubel et al, eds.

The terms “substantially identical” or “substantial identity,” in the context of two or more nucleic acids, refer to two or more sequences or subsequences that are the same or have a specified percentage of nucleotides that are the same (i.e., at least about 60%, preferably at least about 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 75, or 100 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of a number of contiguous positions selected from the group consisting of from about 20 to about 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman 1981, by the homology alignment algorithm of Needleman and Wunsch 1970, by the search for similarity method of Pearson and Lipman 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. (1995 supplement)).

An example of algorithms that are suitable for determining percent sequence identity and sequence similarity are the BLAST and BLAST 2.0 algorithms, which are described in Altschul et al. 1977 and Altschul et al. 1990, respectively. BLAST and BLAST 2.0 are used, with the parameters described herein, to determine percent sequence identity for the disclosed nucleic acids and proteins. Software for performing BLAST analyses is publicly available through the National Center for Biotechnology Information.

The BLAST algorithm also performs a statistical analysis of the similarity between two sequences (see, e.g., Karlin and Altschul 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “nucleic acid” or “polynucleotide” refers to a polymer containing at least two deoxyribonucleotides or ribonucleotides in either single- or double-stranded form and include DNA and RNA. DNA may be in the form of, e.g., antisense oligonucleotides, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, mRNA, tRNA, rRNA, tRNA, vRNA, and combinations thereof. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such modifications are disclosed herein.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

“Systemic delivery,” as used herein, refers to delivery that leads to a broad biodistribution of a compound such as an siRNA within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery can be by any means known in the art including, for example, intravenous, subcutaneous, and intraperitoneal.

“Local delivery,” as used herein, refers to delivery of a compound such as an siRNA directly to a target site within an organism. For example, a compound can be locally delivered by direct injection into a disease site such as a tumor or other target site such as a site of inflammation or a target organ such as the liver, heart, pancreas, kidney, and the like.

The term “mammal” refers to any mammalian species such as a human, mouse, rat, dog, cat, hamster, guinea pig, livestock, and the like. For example, a mammal can be a human.

As described herein, a “subject” can be an animal, e.g., a human being or a mammal. A subject can also be a non-human animal. Examples of a non-human animal include but are not limited to a mouse, rat, pig, monkey, chimpanzee, orangutan, cat, dog, sheep, and cow. A subject can be a natural animal. A subject can also be a transgenic, non-human animal including but not limited to a transgenic mouse or transgenic rat.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic 15 acid), which is assayed as described herein. A sample may also be any body fluid or excretion (for example, but not limited to, blood, urine, stool, saliva, tears, bile) that contains cells or cell components.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed herein are compounds of formula (I):

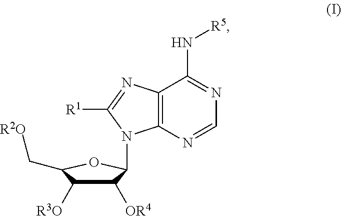

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, optionally substituted aryl, or (viii) substituted heteroaryl; and wherein R² can be a protecting group; and wherein R⁴ can be a protecting group; and wherein R⁵ can be a protecting group. In an aspect, R³ can be a protected phosphate.

Also disclosed herein are compounds of formula (I), wherein R¹ can be OR⁶;

wherein R⁶ can be:

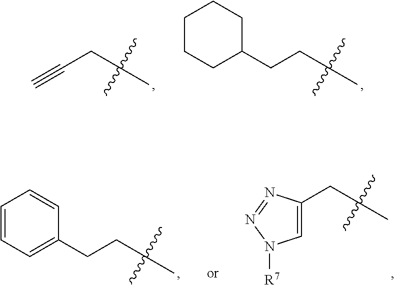

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vii) substituted C1-C12 heteroalkynyl, (viii) substituted aryl, or (ix) substituted heteroaryl. In an aspect, R⁷ can be:

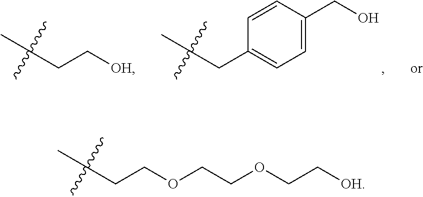

Also disclosed is a compound of Formula I, which can be represented by the formula:

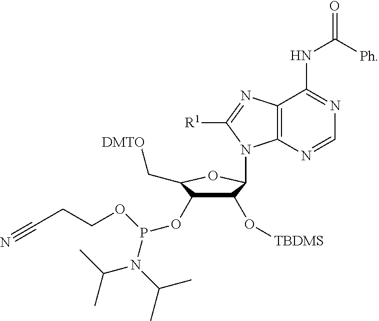

As used herein, “alkyl” refers to a chemical substituent having at least one saturated carbon atom. The alkyl substituents can be linear, branched, or cyclic alkyl. Examples of C₁-C₆ linear or branched alkyl include without limitation methyl (C₁), ethyl (C₂), n-propyl (C₃), iso-propyl (C₃), n-butyl (C₄), sec-butyl (C₄), iso-butyl (C₄), tert-butyl (C₄), pentyl (C₅), iso-pentyl (C₅), hexyl (C₆). The linear or branched alkyl can be substituted or unsubstituted with a variety of substituents, including halogen, hydroxyl, carboxy, amino, amido, cyano, thio, among others. Specific examples of substituted linear or branched include without limitation hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.

Cyclic alkyl groups can comprise rings having from 3 to 20 carbon atoms, wherein the atoms which comprise said rings are limited to carbon atoms, and further each ring can be independently substituted with one or more moieties capable of replacing one or more hydrogen atoms. The following are non-limiting examples of substituted and unsubstituted cyclic alkyl groups which encompass the following categories of units: cyclic rings having a single substituted or unsubstituted hydrocarbon ring, non-limiting examples of which include, cyclopropyl (C₃), 2-methyl-cyclopropyl (C₃), cyclopropenyl (C₃), cyclobutyl (C₄), 2,3-dihydroxycyclobutyl (C₄), cyclobutenyl (C₄), cyclopentyl (C₅), cyclopentenyl (C₅), cyclopentadienyl (C₅), cyclohexyl (C₆), cyclohexenyl (C₆), cycloheptyl (C₇), cyclooctanyl (C₈), decalinyl (C₁₀), 2,5-dimethylcyclopentyl (C₅), 3,5-dichlorocyclohexyl (C₆), 4-hydroxycyclohexyl (C₆), and 3,3,5-trimethylcyclohex-1-yl (C₆); cyclic rings having two or more substituted or unsubstituted fused hydrocarbon rings, non-limiting examples of which include, octahydropentalenyl (C₈), octahydro-1H-indenyl (C₉), 3a,4,5,6,7,7a-hexahydro-3H-inden-4-yl (C₉), decahydroazulenyl (C₁₀); cyclic rings which are substituted or unsubstituted bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, “alkenyl” refers to a chemical substituent having one or more —C═C— double bonds. The alkenyl substituent can be linear, branched, or cyclic alkenyl. Examples of which include without limitation ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

The term “alkynyl” as used herein refers to a subsituents having at least one carbon-carbon triple bond and includes linear, branched, and cyclic alkynyl, non-limiting examples of which include, ethynyl (C₂), prop-2-ynyl (also propargyl) (C₃), propyn-1-yl (C₃), and 2-methyl-hex-4-yn-1-yl (C₂); substituted linear or branched alkynyl, non-limiting examples of which include, 5-hydroxy-5-methylhex-3-ynyl (C₂), 6-hydroxy-6-methylhept-3-yn-2-yl (C₈), 5-hydroxy-5-ethylhept-3-ynyl (C₉), and the like.

Any of the alkyl, alkenyl, or alkyl groups defined above can also comprise heteroatoms within a carbon chain, including for example, O, S, N, or combinations thereof. Thus, ethers, secondary amines, and thiols can be present in any of the above defined groups. Thus, as defined herein, “alkyl” includes groups such as “alkoxy,” including for example, methoxy.

The term “aryl” as used herein refers to a chemical units encompassing at least one phenyl or naphthyl ring and further each ring can be independently substituted with one or more moieties capable of replacing one or more hydrogen atoms. The following are non-limiting examples of “substituted and unsubstituted aryl rings” which encompass the following categories of units: C₆ or C₁₀ substituted or unsubstituted aryl rings; phenyl and naphthyl rings whether substituted or unsubstituted, non-limiting examples of which include, phenyl (C₆), naphthylen-1-yl (C₁₀), naphthylen-2-yl (C₁₀), 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀); C₆ or C₁₀ aryl rings fused with 1 or 2 saturated rings non-limiting examples of which include, bicyclo[4.2.0]octa-1,3,5-trienyl (C₈), and indanyl (C9).

The term “heteroaryl” as used herein includes those units encompassing one or more rings comprising from 5 to 20 atoms wherein at least one atom in at least one ring is a heteroatom chosen from nitrogen (N), oxygen (O), or sulfur (S), or mixtures of N, O, and S, and wherein further at least one of the rings which comprises a heteroatom is an aromatic ring. The following are non-limiting examples of “substituted and unsubstituted heterocyclic rings” which encompass the following categories of units: heteroaryl rings containing a single ring, non-limiting examples of which include, 1,2,3,4-tetrazolyl (C₁), [1,2,3]triazolyl (C₂), [1,2,4]triazolyl (C₂), triazinyl (C₃), thiazolyl (C₃), 1H-imidazolyl (C₃), oxazolyl (C₃), furanyl (C₄), thiopheneyl (C₄), pyrimidinyl (C₄), 2-phenylpyrimidinyl (C₄), pyridinyl (C₅), 3-methylpyridinyl (C₅), and 4-dimethylaminopyridinyl (C₅) heteroaryl rings containing 2 or more fused rings one of which is a heteroaryl ring, non-limiting examples of which include: 7H-purinyl (C₅), 9H-purinyl (C₅), 6-amino-9H-purinyl (C₅), 5H-pyrrolo[3,2-d]pyrimidinyl (C₆), 7H-pyrrolo[2,3-d]pyrimidinyl (C₆), pyrido[2,3-d]pyrimidinyl (C₇), 2-phenylbenzo[d]thiazolyl (C₇), 1H-indolyl (C₈), 4,5,6,7-tetrahydro-1-H-indolyl (C₈), quinoxalinyl (C₈), 5-methylquinoxalinyl (C₈), quinazolinyl (C₈), quinolinyl (C₉), 8-hydroxy-quinolinyl (C₉), and isoquinolinyl (C₉).

The terms “heterocyclic” and/or “heterocycle” as used herein refer to those units comprising one or more rings having from 3 to 20 atoms wherein at least one atom in at least one ring is a heteroatom chosen from nitrogen (N), oxygen (O), or sulfur (S), or mixtures of N, O, and S, and wherein further the ring which comprises the heteroatom is also not an aromatic ring. The following are non-limiting examples of “substituted and unsubstituted heterocyclic rings” which encompass the following categories of units: heterocyclic units having a single ring containing one or more heteroatoms, non-limiting examples of which include, diazirinyl (C₁), aziridinyl (C₂), urazolyl (C₂), azetidinyl (C₃), pyrazolidinyl (C₃), imidazolidinyl (C₃), oxazolidinyl (C₃), isoxazolinyl (C₃), isoxazolyl (C₃), thiazolidinyl (C₃), isothiazolyl (C₃), isothiazolinyl (C₃), oxathiazolidinonyl (C₃), oxazolidinonyl (C₃), hydantoinyl (C₃), tetrahydropyranyl (C₄), pyrrolidinyl (C₄), morpholinyl (C₄), piperazinyl (C₄), piperidinyl (C₄), dihydropyranyl (C₅), tetrahydropyranyl (C₅), piperidin-2-onyl(valerolactam) (C₅), 2,3,4,5-tetrahydro-1H-azepinyl (C₆), 2,3-dihydro-1H-indole (C₈), and 1,2,3,4-tetrahydro-quinoline (C₉); heterocyclic units having 2 or more rings one of which is a heterocyclic ring, non-limiting examples of which include hexahydro-1H-pyrrolizinyl (C₇), 3a,4,5,6,7,7a-hexahydro-1H-benzo[d]imidazolyl (C₇), 3a,4,5,6,7,7a-hexahydro-1H-indolyl (C₈), 1,2,3,4-tetrahydroquinolinyl (C₉), and decahydro-1H-cycloocta[b]pyrrolyl (C₁₀).

The term “halide” is intended to refer to Br, Cl, I, and F.

The term “amino” refers to any substituted or unsubstituted primary, secondary, or tertiary amine.

The term “substituted” is used throughout the specification. The term “substituted” is applied to the units described herein as a substituted unit or moiety which has one or more hydrogen atoms replaced by a substituent or several substituents as defined herein below. The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring (aryl ring)”, (N,N-dimethyl-5-amino)octanyl is a “substituted C₈ linear alkyl unit, 3-guanidinopropyl is a “substituted C₃ linear alkyl unit,” and 2-carboxypyridinyl is a “substituted heteroaryl unit.”

The following are non-limiting examples of units which can be substituents on a residue or chemical moiety that is defined as substituted: i) C1-C12 linear, branched, or cyclic alkyl, alkenyl, and alkynyl; methyl (C1), ethyl (C2), ethenyl (C2), ethynyl (C2), n-propyl (C3), iso-propyl (C3), cyclopropyl (C3), 3-propenyl (C3), 1-propenyl (also 2-methylethenyl) (C3), isopropenyl (also 2-methylethen-2-yl) (C3), prop-2-ynyl (also propargyl) (C3), propyn-1-yl (C3), n-butyl (C4), sec-butyl (C4), iso-butyl (C4), tert-butyl (C4), cyclobutyl (C4), buten-4-yl (C4), cyclopentyl (C5), cyclohexyl (C6); ii) substituted or unsubstituted C6 or C10 aryl; for example, phenyl, naphthyl (also referred to herein as naphthylen-1-yl (C10) or naphthylen-2-yl (C10)); iii) substituted or unsubstituted C6 or C10 alkylenearyl; for example, benzyl, 2-phenylethyl, naphthylen-2-ylmethyl; iv) substituted or unsubstituted C1-C9 heterocyclic rings; as described herein; v) substituted or unsubstituted C1-C9 heteroaryl rings; as described herein; vi) —(CR102aR102b)aOR101; for example, —OH, —CH₂OH, —OCH₃, —CH₂OCH3, —OCH₂CH₃, —CH₂OCH₂CH3, —OCH₂CH₂CH₃, and —CH₂OCH₂CH₂CH₃; vii) —(CR102aR102b)aC(O)R101; for example, —COCH₃, —CH₂COCH₃, —OCH₂CH₃, —CH₂COCH₂CH₃, —COCH₂CH₂CH₃, and —CH₂COCH₂CH₂CH₃; vii) —(CR102aR102b)aC(O)OR101; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃; —(CR102aR102b)aC(O)N(R101)₂; for example, —CONH₂, —CH₂CONH₂, —CONHCH₃, —CH₂CONHCH₃, —CON(CH₃)₂, and —CH₂CON(CH₃)₂; —(CR102aR102b)aN(R101)₂; for example, —NH2, —CH₂NH₂, —NHCH₃, —CH₂NHCH₃, —N(CH₃)₂, and —CH₂N(CH₃)₂; halogen; —F, —Cl, —Br, and —I; —(CR102aR102b)aCN; —(CR102aR102b)aNO₂; —CHjXk; wherein X is halogen, the index j is an integer from 0 to 2, j+k=3; for example, —CH₂F, —CF₃, —CCl₃, or —CBr₃; (CR102aR102b)aSR101; —SH, —CH₂SH, —SCH₃, —CH₂SCH₃, —SC₆H₅, and —CH₂SC₆H₅; —(CR102aR102b)aSO₂R101; for example, and —SO₂H, —CH₂SO₂H, —SO₂CH₃, —CH₂SO₂CH₃, —SO₂C₆H₅, and —CH₂SO₂C₆H₅; and —(CR102aR102b)aSO₃R101; for example, —SO₃H, —CH₂SO₃H, —SO₃CH₃, —CH₂SO₃CH₃, —SO₃C₆H₅, and —CH₂SO₃C₆H₅; wherein each R101 is independently hydrogen, substituted or unsubstituted C₁-C₄ linear, branched, or cyclic alkyl, phenyl, benzyl, heterocyclic, or heteroaryl; or two R101 units can be taken together to form a ring comprising 3-7 atoms; R102a and R102b are each independently hydrogen or C1-C4 linear or branched alkyl; the index “a” is from 0 to 4.

Also disclosed herein are nucleosides. In an aspect, the disclosed nucleoside can be represented by Formula II:

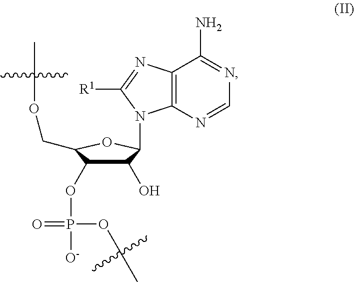

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, (viii) substituted aryl, or (ix) substituted heteroaryl. In an aspect, R¹ can be chloride. In an aspect, R¹ can be OR⁶; wherein R⁶ can be:

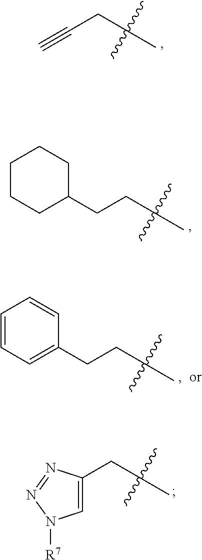

and
R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (ix) substituted heteroaryl. In an aspect, R⁷ can be:

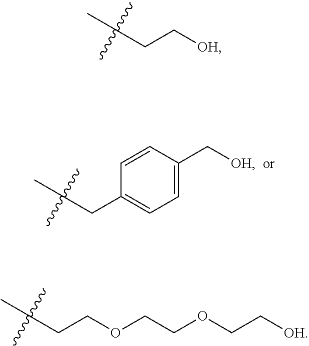

Also disclosed herein are oligonucleotides or polynucleotides comprising at least one nucleoside of Formula II:

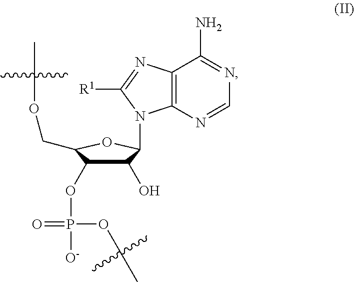

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, (viii) substituted aryl, or (ix) substituted heteroaryl. In as aspect, R⁶ of the disclosed oligonucleotide or polynucleotide can be:

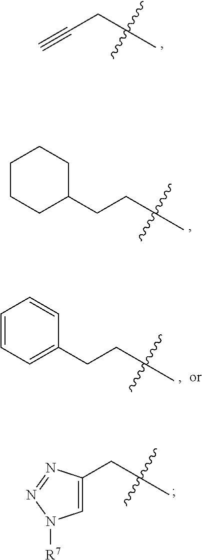

and
R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (viii) substituted heteroaryl. In as aspect, R⁷ can be:

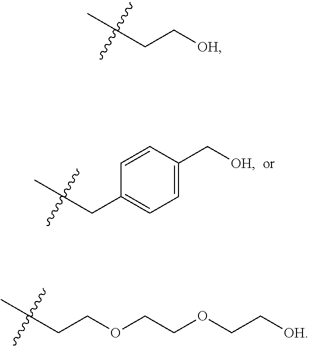

The disclosed compounds can be generally represented by formula (I):

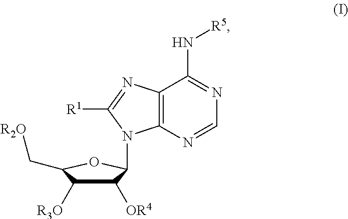

wherein R¹ is selected from halide and OR⁶, wherein R⁶ is selected from optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, and optionally substituted heteroaryl; wherein each of R², R⁴, and R⁵ independently comprises a protecting group; and wherein R³ comprises a protected phosphate.

The substituent at R¹ can create a steric blockade in the minor and major grooves of the disclosed dsRNAs and can reduce or eliminate unwanted binding of proteins to the dsRNA. The efficiency of binding between the siRNA and the target sequence can be improved. Generally, the substituent at R1 can preferably be one which can allow the residue of formula (II) in the siRNA to bind to a complementary base in the duplex to create a Hoogsteen base pair. Thus, R1 can be preferably attached to the adenosine through an electronegative atom, such as oxygen or chloride, which can allow for such a Hoogsteen pair to form. The dsRNA can be delivered to a target mRNA in such a duplex arrangement.

As the dsRNA binds to a target mRNA, the residue of formula (II) can shift into an anti conformation and bind, for example, to a uracil residue in the target mRNA such that R¹ creates a steric hindrance in the major groove of the resulting mRNA-antisense strand duplex.

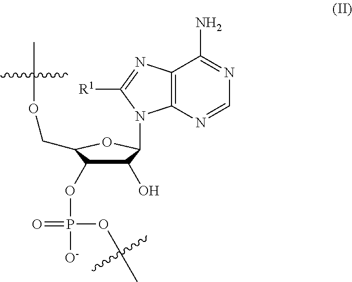

The substituent at R¹ can comprise a variety of residues that provide for a steric blockade in the major groove of a strand duplex. The switch to the anti conformation can also relieve steric interactions with proteins and can permit activity in the RISC for effective RNA interference.

In one aspect, R1 is chloride. In a further aspect, R1 is OR⁶ wherein R⁶ is selected from optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, and optionally substituted heteroaryl.

In one aspect, R⁶ can be selected from:

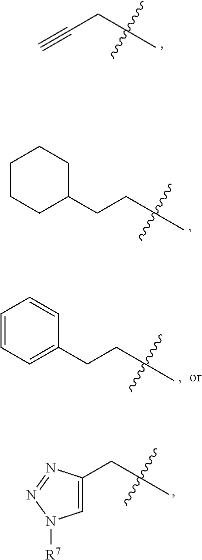

wherein R⁷ is selected from optionally substituted C1-C12 alkyl, optionally substituted C1-C12 heteroalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, and optionally substituted heteroaryl.

When R⁶ comprises propargyl, the compound can be further modified with a variety of substituents through “click chemistry” with a corresponding azide. Methods for carrying out “click” reactions are known in the art.

Protecting groups within R²-R⁵ can be any protecting group commonly used in DNA or RNA synthesis, which will allow for the compound of formula (I) to be incorporated into the antisense strand. In some aspects, R² and R³ or R³ and R⁴ can be covalently bonded to one another to form a ring together with the atoms to which they are attached.

Suitable protecting groups as substituents R²-R⁴ include common hydroxyl protecting groups, such as acetyl (Ac) (removed by acid or base); benzoyl (Bz) —(removed by acid or base, more stable than Ac group); benzyl (Bn, Bnl) (removed by hydrogenolysis); β-methoxyethoxymethyl ether (MEM) (removed by acid); dimethoxytrityl [bis-(4-methoxyphenyl)phenylmethyl, (DMT) (removed by weak acid); methoxymethyl ether (MOM) (removed by acid); methoxytrityl[(4-methoxyphenyl)diphenylmethyl, MMT) (removed by acid and hydrogenolysis); p-Methoxybenzyl ether (PMB) (removed by acid, hydrogenolysis, or oxidation); methylthiomethyl ether (removed by acid); pivaloyl (Piv) (removed by acid, base or reducing agents); tetrahydropyranyl (THP) (removed by acid); trityl (triphenylmethyl, Tr) (removed by acid and hydrogenolysis); silyl ether (e.g., trimethylsilyl (TMS), tert-butyldimethylsilyl (TBDMS), tert-butyldimethylsilyloxymethyl (TOM), and triisopropylsilyl (TIPS) ethers) (removed by acid or fluoride ion (such as NaF, TBAF (Tetra-n-butylammonium fluoride, HF-Py, or HF-NEt3); methyl ethers (cleavage is by TMSI in DCM or MeCN or chloroform or is BBr3 in DCM); and ethoxyethyl ethers (EE) (cleavage by, e.g., 1N hydrochloric acid).

Suitable protecting groups as a substitent R⁵ include common amine protecting groups, such as carbobenzyloxy (Cbz) (removed by hydrogenolysis); p-Methoxybenzyl carbonyl (Moz or MeOZ) (removed by hydrogenolysis); tert-Butyloxycarbonyl (BOC) group (removed by concentrated, strong acid. (such as HCl or CF₃COOH); 9-fluorenylmethyloxycarbonyl (FMOC) (removed by base, such as piperidine); benzyl (Bn) group (removed by hydrogenolysis); p-Methoxybenzyl (PMB) (removed by hydrogenolysis); 3,4-Dimethoxybenzyl (removed by hydrogenolysis); (p-methoxyphenyl (PMP) (removed by Ammonium cerium (IV) nitrate (CAN)); tosyl (Ts) (removed by concentrated acid (HBr, H₂SO4) and strong reducing agents (sodium in liquid ammonia, sodium napthalene)); and other sulfonamides (Nosyl & Nps) (removed by samarium iodide, tributyltin hydride).

Substituent R³ comprises a protected phosphate, such as a phosphate protected with a cyanoethyl protecting group (removed by mild base), or diisopropylamine.

The compound below is a specific example of a compound of formula (I):

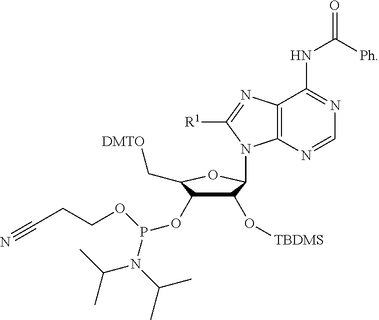

In this example, R² is protected with a dimethoxytrityl[bis-(4-methoxyphenyl)phenylmethyl, (DMT) group, R⁴ is protected with a tert-butyl dimethylsilyl (TBDMS) group, R⁵ is protected with a benzyl group, and R³ comprises a phosphate which is protected with a 2-cyanoethyl group and diisopropylamine.

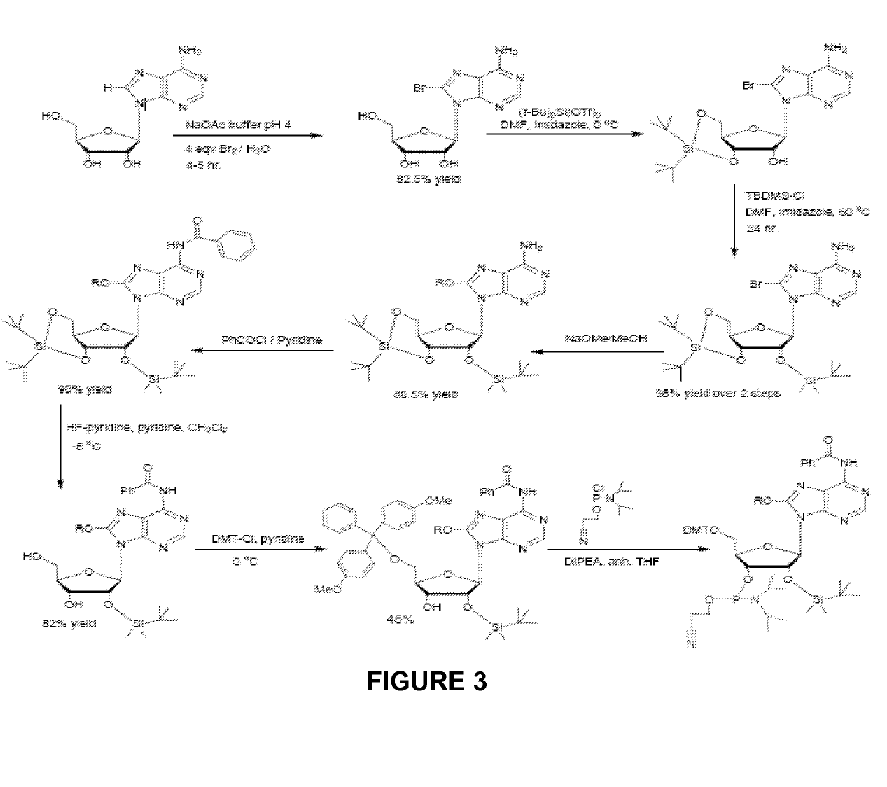

A representative synthesis of the compounds of formula (I) is shown in FIG. 3. With reference to FIG. 3, the starting bromoadenosine can be provided using a three to four-fold excess of bromine in the presence of adenosine. In one aspect, di-t-butylsilyl ditriflate (DTBSDT) can be used to protect the 5′- & 3′-OH simultaneously, leaving the 2′-OH ready to be protected by TBDMS-Cl. These two reactions can be carried out in one-port. R¹ can be attached with a suitable alkoxy group in the presence of a strong base, such as NaOMe. The deprotection of 5′- & 3′-OH was carried out by using a fluoride reagent, HF-pyridine, at sub-zero temperature. The phosphoramidite synthesis step is carried out using conventional methods. The modified adenosine phosphoramidite can then be incorporated into the antisense strand of dsRNA.

The disclosed RNAs can comprise the RNA-incorporated analog of the compound of formula (I), after the compound has been incorporated into the RNA through conventional nucleotide synthesis. Disclosed herein are RNAs, for example double-stranded RNA (dsRNA) comprising a sense region and an antisense region that together form a duplex having from 15 to 60 base pairs; wherein the antisense strand comprises a sequence sufficiently complementary to a target mRNA sequence to direct target-specific RNA interference (RNAi); and wherein the antisense region comprises one or more residues of formula (II):

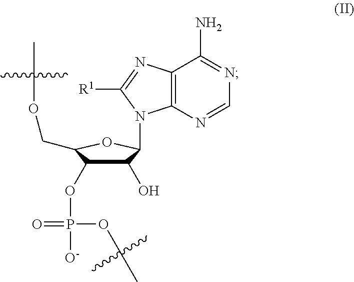

wherein R¹ is selected from halide and OR⁶, wherein R⁶ is selected from optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, and optionally substituted heteroaryl. The substituent R¹, which provides the steric blockade as discussed above, can include any of those substituents discussed above in reference to the starting material protected adenosine analogs of formula (I).

Disclosed herein are antisense RNAs capable of silencing expression of a target sequence. The antisense RNA can comprise from about 18 to about 38 nucleotides. For example, disclosed are antisense RNAs that comprise from about 15 to about 30 nucleotides.

Disclosed herein are antisense RNAs comprising at least one modified adenosine as described herein. The modified adenosine can be present in one strand (i.e., sense or antisense) or both strands of the siRNA. The antisense RNA sequences can have overhangs (e.g., 3′ or 5′ overhangs as described in Elbashir et al. 2001 or Nykanen et al. 2001, or may lack overhangs (i.e., have blunt ends).

According to the methods described herein, antisense RNA can be modified to decrease their off-target interactions without having a negative impact on RNAi activity. For example, a modified interfering RNA can be capable of silencing expression of the target sequence. This can lead to increased interfering RNA activity. Suitable interfering RNA sequences can be identified using any means known in the art. Typically, the methods described in Elbashir et al. 2001 and Elbashir et al. 2001 can be combined with rational design rules set forth in Reynolds et al. 2004. Generally, the sequence within about 50 to about 100 nucleotides 3′ of the AUG start codon of a transcript from the target gene of interest is scanned for dinucleotide sequences (e.g., AA, CC, GG, or UU) (see, e.g., Elbashir et al. 2001). The nucleotides immediately 3′ to the dinucleotide sequences are identified as potential interfering RNA target sequences. Typically, the 19, 21, 23, 25, 27, 29, 31, 33, 35, or more nucleotides immediately 3′ to the dinucleotide sequences are identified as potential siRNA target sites. In some embodiments, the dinucleotide sequence is an AA sequence and the 19 nucleotides immediately 3′ to the AA dinucleotide are identified as a potential siRNA target site. Interfering RNA target sites can be spaced at different positions along the length of the target gene. To further enhance silencing efficiency of the interfering RNA sequences, potential interfering RNA target sites may be further analyzed to identify sites that do not contain regions of homology to other coding sequences. For example, a suitable interfering RNA target site of about 21 base pairs typically will not have more than 16-17 contiguous base pairs of homology to other coding sequences. If the interfering RNA sequences are to be expressed from an RNA Pol III promoter, interfering RNA target sequences lacking more than 4 contiguous A's or T's are selected.

Once the potential interfering RNA target site has been identified, interfering RNA sequences complementary to the interfering RNA target sites may be designed. To enhance their silencing efficiency, the interfering RNA sequences may also be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. Interfering RNA design tools that incorporate algorithms that assign suitable values of each of these features and are useful for selection of interfering RNA can be found at Ambion Technical Bulletin No. 506 (http://www.ambion.com/techlib/tb/tb_{—}506.html) and Yuan et al., 2004. Interfering RNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 3′ or 5′ overhangs as described in Elbashir et al. 2001 or Nykanen et al. 2001, or may lack overhangs (i.e., to have blunt ends).

An RNA population can be used to provide long precursor RNAs, or long precursor RNAs that have substantial or complete identity to a selected target sequence can be used to make the interfering RNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement can also be transcribed in vitro and hybridized to form a dsRNA. If a naturally occurring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs can then hybridized to form double stranded RNAs for digestion. The dsRNAs can be directly administered to a subject or can be digested in vitro prior to administration.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use disclosed herein include Sambrook et al., Molecular Cloning, A Laboratory Manual (2nd ed. 1989); Kriegler, Gene Transfer and Expression. A Laboratory Manual (1990); and Current Protocols in Molecular Biology (Ausubel et al., eds., 1994).

Disclosed herein are methods of blocking binding of an off-target molecule to an interfering RNA molecule, the method comprising modifying at least one adenosine of the interfering RNA molecule. The interfering RNA can comprise two or more modified adenosines. Examples of modified bases are described herein. The off-target molecule can be any double stranded RNA-binding motif (dsRBM). For example, the off-target molecule can be PKR or ADAR or 2′,5′-oligoadenylate synthase (OAS). The off-target molecule can also be Toll-Like Receptor-3, Toll-Like Receptor-7, Toll-Like Receptor-8, or Toll-Like Receptor-9.

The term “blocking” refers to inhibiting the interaction between siRNA and an off-target molecule. For example, the interaction between an off-target molecule and the modified interfering RNA can be inhibited or reduced by 10, 20, 30, 40, 50, 60, 70, 80, 90, or 100%, or any amount in between.

By “off-target molecule” is meant a molecule other than the target intended to interact with the siRNA molecule. This can be any molecule at all that may come into contact with the siRNA that is not the intended target.

The disclosed nucleobases can be incorporated into a nucleic acid strand using methods known in the art. To incorporate the nucleobase into a nucleic acid strand, R³ will typically be a cyclic moiety, such as a sugar moiety, as discussed above which has attached thereto a nucleic acid coupling agent. Numerous examples are known in the art, including phosphodiesters, phosphotriesters, phosphate trimesters, phosphonates, phosphoramidites, among others. For a detailed explanation of how to incorporate the nucleobases into a nucleic acid strand, see Blackburn and Williams 2006, which is incorporated herein by this reference for its teaching of methods for incorporating nucleobases into nucleic acid strands. When incorporating the disclosed nucleobases in strands of nucleic acids, it can be useful to protect vulnerable groups, for example hydroxyl groups with a suitable protecting group.

Thus, it certain embodiments, it can be desirable to protect R⁴ when R⁴ is present as a hydroxyl group. Likewise, when R⁶ is present, R⁶ can comprise a suitable protecting group as desired. A wide variety of hydroxyl protecting groups can be used. Representative hydroxyl protecting groups are disclosed by Beaucage et al. 1992, and also in e.g., Green and Wuts 1991, both of which are incorporated herein by this reference, for their teachings of hydroxyl protecting groups. Specific examples of hydroxyl protecting include dimethoxytrityl (DMT), monomethoxytrityl, 9-phenylxanthen-9-yl (Pixyl) and 9-(p-methoxyphenyl)xanthen-9-yl (Mox). Other examples include various silyl ethers, such as tert-butyl dimethyl silyl either (TBDMS).

The protecting groups can be removed as desired, for example after the nucleobase has been incorporated into a strand of DNA or RNA. The R⁶ or R⁴ protecting group, when present, for example, can be removed by techniques well known in the art to form the free hydroxyl group. For example, dimethoxytrityl (DMT) protecting groups can be removed by protic acids such as formic acid, dichloroacetic acid, trichloroacetic acid, p-toluene sulphonic acid or with a Lewis acid such as zinc bromide.

The nucleobases disclosed herein can be made using a variety of methods known to the art.

The disclosed modified interfering RNA molecules can be synthesized via a tandem synthesis technique, wherein both strands are synthesized as a single continuous oligonucleotide fragment or strand separated by a cleavable linker that is subsequently cleaved to provide separate fragments or strands that hybridize to form the interfering RNA duplex. The linker can be a polynucleotide linker or a non-nucleotide linker. The tandem synthesis of modified interfering RNA can be readily adapted to both multiwell/multiplate synthesis platforms as well as large scale synthesis platforms employing batch reactors, synthesis columns, and the like. Alternatively, the disclosed modified interfering RNA molecules can be assembled from two distinct oligonucleotides, wherein one oligonucleotide comprises the sense strand and the other comprises the antisense strand of the interfering RNA. For example, each strand can be synthesized separately and joined together by hybridization or ligation following synthesis and/or deprotection. In certain other instances, the modified interfering RNA molecules can be synthesized as a single continuous oligonucleotide fragment, where the self-complementary sense and antisense regions hybridize to form an interfering RNA duplex having hairpin secondary structure.

In certain embodiments, in addition to the modified adenosines, the disclosed interfering RNA molecules further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., 1993). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al. 1995; Mesmaeker et al. 1994). Such chemical modifications can occur at the 5′-end and/or 3′-end of the sense strand, antisense strand, or both strands of the siRNA.

In other embodiments, chemical modification of the interfering RNA comprises attaching a conjugate to the chemically-modified interfering RNA molecule. The conjugate can be attached at the 5′ and/or 3′-end of the sense and/or antisense strand of the chemically-modified interfering RNA via a covalent attachment such as, e.g., a biodegradable linker. The conjugate can also be attached to the chemically-modified interfering RNA, e.g., through a carbamate group or other linking group (see, e.g., U.S. Patent Publication Nos. 20050074771, 20050043219, and 20050158727). In certain instances, the conjugate is a molecule that facilitates the delivery of the chemically-modified interfering RNA into a cell. Examples of conjugate molecules suitable for attachment to the chemically-modified interfering RNA disclosed herein include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325.

Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the chemically-modified interfering RNA molecule can be evaluated for improved pharmacokinetic profiles, bioavailability, and/or stability of the interfering RNA while retaining full RNAi activity. As such, one skilled in the art can screen chemically-modified interfering RNA molecules having various conjugates attached thereto to identify ones having improved properties and full RNAi activity using any of a variety of well-known in vitro cell culture or in vivo animal models.

### C. METHODS

Also disclosed herein are methods of blocking the binding of an off-target molecule to an siRNA molecule, comprising, modifying at least one adenosine of the siRNA molecule, and administering to a subject the siRNA molecule. For example, disclosed herein are methods of blocking the binding of an off-target molecule to an siRNA molecule, comprising, modifying at least one adenosine of the siRNA molecule, wherein the siRNA molecule comprises one or more modified adenosines, and administering to a subject the siRNA molecule.

Also disclosed herein are methods of blocking the binding of an off-target molecule to an siRNA molecule, comprising, modifying at least two adenosines of the siRNA molecule, and administering to a subject the siRNA molecule. For example, disclosed herein are methods of blocking the binding of an off-target molecule to an siRNA molecule, comprising, modifying at least one adenosine of the siRNA molecule, wherein the siRNA molecule comprises two or more modified adenosines, and administering to a subject the siRNA molecule.

Also disclosed herein are methods of blocking the binding of an off-target molecule to an siRNA molecule, comprising, modifying at least three adenosines of the siRNA molecule, and administering to a subject the siRNA molecule. For example, disclosed herein are methods of blocking the binding of an off-target molecule to an siRNA molecule, comprising, modifying at least one adenosine of the siRNA molecule, wherein the siRNA molecule comprises three or more modified adenosines, and administering to a subject the siRNA molecule.

Also disclosed herein are methods of blocking the binding of an off-target molecule to an siRNA molecule, comprising, modifying at least one adenosine base of the siRNA molecule, and administering to a subject the siRNA molecule, wherein the modified adenosine comprises Formula I:

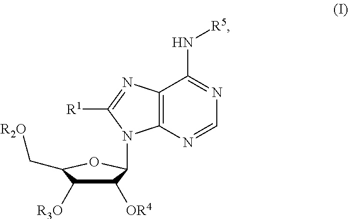

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, optionally substituted aryl, or (viii) substituted heteroaryl; and wherein R² can be a protecting group; and wherein R⁴ can be a protecting group; and wherein R⁵ can be a protecting group. In an aspect, R³ can be a protected phosphate. In an aspect, R¹ can be OR⁶ and wherein R⁶ can be:

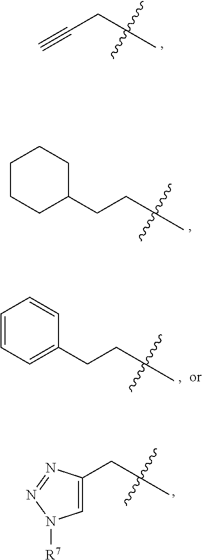

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vii) substituted C1-C12 heteroalkynyl, (viii) substituted aryl, or (ix) substituted heteroaryl. In an aspect, R⁷ can be:

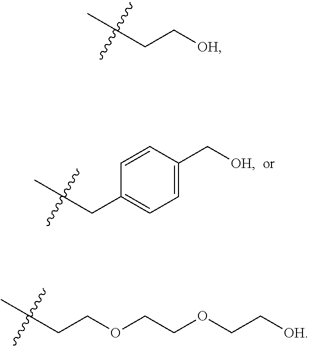

Also disclosed herein are methods of blocking the binding of an off-target molecule to an siRNA molecule, comprising, modifying at least one adenosine of the siRNA molecule, and administering to a subject the siRNA molecule, wherein the off-target molecule is a double stranded RNA-binding motif (dsRBM).

In addition, certain siRNAs have been shown to activate the innate immune response in mammalian cells in a sequence-specific manner and are believed to occur via Toll-like receptor 7 (TLR7) present in the endosomal membrane. It appears that molecular recognition by TLR7 occurs by TLR7 making contact to the bases similar to other RNA binding proteins (Elliott et al. 1999). Disclosed herein are compositions and methods comprising modified bases that inhibit binding to TLR3, TLR7, TLR8, TLR9, and related immunostimulatory proteins. For example, disclosed herein are compositions and methods comprising modified bases that inhibit binding to TLR7 and related immunostimulatory proteins, wherein at least one adenosine of an siRNA molecule has been modified or altered.

Also disclosed herein are methods of blocking the binding of an off-target molecule to an interfering RNA molecule, comprising, modifying at least one adenosine of the interfering RNA molecule, and administering to a subject the interfering RNA molecule, wherein the off-target molecule is a double stranded RNA-binding motif (dsRBM). For example, dsRBMs include but are not limited to RNA dependent protein kinase (PKR), adenosine deaminase (ADAR), 2′5,-oligoadenylate synthase (OAS), Toll-Like Receptor-3, Toll-Like Receptor-7, Toll-Like Receptor-8, and Toll-Like Receptor-9.

The interfering RNA described herein can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation, angiogenic genes, immunomodulator genes such as those associated with inflammatory and autoimmune responses, ligand receptor genes, and genes associated with neurodegenerative disorders.

Selective incorporation of modified adenosines into either strand of the interfering RNA duplex can reduce or completely abrogate the off-target response to synthetic interfering RNA. Modified interfering RNA targeting can mediate potent silencing of its target sequence. Advantageously, the approach to interfering RNA design and delivery described herein is widely applicable and advances synthetic interfering RNA into a broad range of therapeutic areas. For example, disclosed herein is a method of synthesizing 8-alkyoxyadenosine as cyanoethylphosphoramidites wherein “alkyl” is propargyl, phenethyl, or cyclohexylethyl, or, for the purposes of comparison, hydrogen and wherein many other alkyl groups can be envisioned by the same synthetic route. The modified adenosines, X, are individually incorporated into RNA oligonucleotides at one or more positions in which a single X:U base pair replaces a A:U base pair in the antisense:sense duplex. The X:G base pair can replace the A:U base pair in an antisense:sense siRNA duplex so that the duplex obtained at the RISC can be an X:U base pair (antisense:mRNA). The modified adenosine X can also be placed in the sense strand. In this case, a G:X base pair can replace a G:C base pair (antisense:sense).

Accordingly, in an aspect, the compositions disclosed herein relate to a pharmaceutical composition comprising a modified interfering RNA according to the disclosed methods and compositions and a pharmaceutically acceptable diluent, carrier or adjuvant. In another aspect, the disclosed modified interfering RNA can be used as a medicament.

As will be understood dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual interfering RNAs. Generally it can be estimated based on EC50s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

As indicated above, the disclosed compositions can relate to a pharmaceutical composition, which comprises at least one modified interfering RNA disclosed herein as an active ingredient. It should be understood that the disclosed pharmaceutical composition optionally comprises a pharmaceutical carrier, and that the pharmaceutical composition optionally comprises further compounds, such as chemotherapeutic compounds, anti-inflammatory compounds, antiviral compounds and/or immuno-modulating compounds.

The disclosed modified interfering RNAs can be used “as is” or in form of a variety of pharmaceutically acceptable salts. As used herein, the term “pharmaceutically acceptable salts” refers to salts that retain the desired biological activity of the herein-identified modified interfering RNAs and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine.

In an aspect, the modified interfering RNA can be in the form of a pro-drug. Oligonucleotides are by virtue negatively charged ions. Due to the lipophilic nature of cell membranes the cellular uptake of oligonucleotides are reduced compared to neutral or lipophilic equivalents. This polarity “hindrance” can be avoided by using the pro-drug approach (see, e.g., Crooke 1998). In this approach the oligonucleotides are prepared in a protected manner so that the oligo is neutral when it is administered. These protection groups are designed in such a way that they can be removed when the oligo is taken up by the cells. Examples of such protection groups are S-acetylthioethyl (SATE) or S-pivaloylthioethyl (t-butyl-SATE). These protection groups are nuclease resistant and are selectively removed intracellulary.

Pharmaceutically acceptable binding agents and adjuvants may comprise part of the formulated drug. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion. A disclosed compound can be mixed with any material that do not impair the desired action, or with material that supplement the desired action. These could include other drugs including other nucleotide compounds. For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline. Preferably, an oligomeric compound is included in a unit formulation such as in a pharmaceutically acceptable carrier or diluent in an amount sufficient to deliver to a patient a therapeutically effective amount without causing serious side effects in the treated patient.

The disclosed pharmaceutical compositions can be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In an aspect, the pharmaceutical composition is administered IV, IP, orally, topically or as a bolus injection or administered directly in to the target organ. Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the disclosed compounds are in admixture with a topical delivery agent such as lipids, liposomes, fatty acids, fatty acid esters, steroids, chelating agents and surfactants. Compositions and formulations for oral administration include but is not restricted to powders or granules, microparticulates, nanoparticulates, suspensions or solutions in water or non-aqueous media, capsules, gel capsules, sachets, tablets or minitablets. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The disclosed pharmaceutical compositions include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to tumour tissue may be enhanced by carrier-mediated delivery including, but not limited to, cationic liposomes, cyclodextrins, porphyrin derivatives, branched chain dendrimers, polyethylenimine polymers, nanoparticles and microspheres (Dass 2002). The pharmaceutical formulations disclosed herein can conveniently be presented in unit dosage form, may be prepared according to conventional techniques well known in the pharmaceutical industry. Such techniques include the step of bringing into association the active ingredients with the pharmaceutical carriers) or excipient(s). In general the formulations are prepared by uniformly and intimately bringing into association the active ingredients with liquid carriers or finely divided solid carriers or both, and then, if necessary, shaping the product. The disclosed compositions can be formulated into any of many possible dosage forms such as, but not limited to, tablets, capsules, gel capsules, liquid syrups, soft gels and suppositories. The compositions can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The compounds can also be conjugated to active drug substances, for example, aspirin, ibuprofen, a sulfa drug, an antidiabetic, an antibacterial or an antibiotic.

The compounds disclosed herein are useful for a number of therapeutic applications as indicated above. In general, the disclosed therapeutic methods include administration of a therapeutically effective amount of a modified interfering RNA to a mammal, particularly a human. In an aspect, disclosed herein are pharmaceutical compositions comprising (a) one or more disclosed compounds, and (b) one or more chemotherapeutic agents. When used with the disclosed compounds, such chemotherapeutic agents may be used individually, sequentially, or in combination with one or more other such chemotherapeutic agents or in combination with radiotherapy. All chemotherapeutic agents known to a person skilled in the art are here incorporated as combination treatments with the disclosed compounds. Other active agents, such as anti-inflammatory drugs, including but not limited to nonsteroidal anti-inflammatory drugs and corticosteroids, antiviral drugs, and immuno-modulating drugs may also be combined in compositions. Two or more combined compounds may be used together or sequentially.

The use of a modified interfering RNA disclosed herein can also be used for the manufacture of a medicament for the treatment of cancer. In another aspect, disclosed herein are methods for treatment of, or prophylaxis against, cancer, said method comprising administering a modified interfering RNA or a pharmaceutical composition comprising a modified interfering RNA to a patient in need thereof.

Such cancers may include lymphoreticular neoplasia, lymphoblastic leukemia, brain tumors, gastric tumors, plasmacytomas, multiple myeloma, leukemia, connective tissue tumors, lymphomas, and solid tumors.

The disclosed compounds can also be used in the manufacture of a medicament for the treatment of cancer, said cancer may suitably be in the form of a solid tumor. Analogously, in the method for treating cancer disclosed herein said cancer may suitably be in the form of a solid tumor.

Furthermore, said cancer is also suitably a carcinoma. The carcinoma is typically selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors. More typically, said carcinoma is selected from the group consisting of malignant melanoma, non-small cell lung cancer, breast carcinoma, colon carcinoma and renal cell carcinoma. The malignant melanoma is typically selected from the group consisting of superficial spreading melanoma, nodular melanoma, lentigo maligna melanoma, acral melagnoma, amelanotic melanoma and desmoplastic melanoma.

Alternatively, the cancer may suitably be a sarcoma. The sarcoma is typically in the form selected from the group consisting of osteosarcoma, Ewing's sarcoma, chondrosarcoma, malignant fibrous histiocytoma, fibrosarcoma and Kaposi's sarcoma. Alternatively, the cancer may suitably be a glioma.

Also disclosed is a method of using a modified interfering RNA disclosed herein for the manufacture of a medicament for the treatment of cancer, wherein said medicament further comprises a chemotherapeutic agent selected from the group consisting of adrenocorticosteroids, such as prednisone, dexamethasone or decadron; altretamine (hexylen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, the further chemotherapeutic agent is selected from taxanes such as Taxol, Paclitaxel or Docetaxel.

Similarly, also disclosed is the use of a modified interfering RNA as described herein for the manufacture of a medicament for the treatment of cancer, wherein said treatment further comprises the administration of a further chemotherapeutic agent selected from the group consisting of adrenocorticosteroids, such as prednisone, dexamethasone or decadron; altretamine (hexylen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, said treatment further comprises the administration of a further chemotherapeutic agent selected from taxanes, such as Taxol, Paclitaxel or Docetaxel.

Herein disclosed are methods for treating cancer, comprising administering a modified interfering RNA as disclosed herein or a pharmaceutical composition comprising modified interfering RNA to a patient in need thereof and further comprising the administration of a further chemotherapeutic agent. Said further administration may be such that the further chemotherapeutic agent is conjugated to a disclosed compound, is present in the pharmaceutical composition, or is administered in a separate formulation.

The disclosed modified interfering RNA compounds can be used for targeting Severe Acute Respiratory Syndrome (SARS), which first appeared in China in November 2002. According to the WHO over 8,000 people have been infected world-wide, resulting in over 900 deaths. A previously unknown coronavirus has been identified as the causative agent for the SARS epidemic (Drosten et al. 2003; Fouchier et al. 2003). Identification of the SARS-COV was followed by rapid sequencing of the viral genome of multiple isolates (Ruan et al. 2003; Rota et al. 2003; Marra 2003). This sequence information immediately made possible the development of SARS antivirals by nucleic acid-based knock-down techniques such as interfering RNA. The nucleotide sequence encoding the SARS-COV RNA-dependent RNA polymerase (Pol) is highly conserved throughout the coronavirus family. The Pol gene product is translated from the genomic RNA as a part of a polyprotein, and uses the genomic RNA as a template to synthesize negative-stranded RNA and subsequently sub-genomic mRNA. The Pol protein is thus expressed early in the viral life cycle and is crucial to viral replication.

The modified interfering RNA disclosed herein can also be used for the manufacture of a medicament for the treatment of Severe Acute Respiratory Syndrome (SARS), as well as to a method for treating Severe Acute Respiratory Syndrome (SARS), said method comprising administering a modified interfering RNA as disclosed or a pharmaceutical composition comprising a modified interfering RNA to a patient in need thereof.

The disclosed compounds and compositions can be broadly applied to a range of infectious diseases, such as diphtheria, tetanus, pertussis, polio, hepatitis B, hemophilus influenza, measles, mumps, and rubella.

Also disclosed is the use of the modified interfering RNA described herein for the manufacture of a medicament for the treatment of an infectious disease, as well as to a method for treating an infectious disease, said method comprising administering a modified interfering RNA or a pharmaceutical composition to a patient in need thereof.

The inflammatory response is an essential mechanism of defense of the organism against the attack of infectious agents, and it is also implicated in the pathogenesis of many acute and chronic diseases, including autoimmune disorders. In spite of being needed to fight pathogens, the effects of an inflammatory burst can be devastating. It is therefore often necessary to restrict the symptomatology of inflammation with the use of anti-inflammatory drugs. Inflammation is a complex process normally triggered by tissue injury that includes activation of a large array of enzymes, the increase in vascular permeability and extravasation of blood fluids, cell migration and release of chemical mediators, all aimed to both destroy and repair the injured tissue.

In an aspect, disclosed herein is a method of using modified interfering RNA as disclosed for the manufacture of a medicament for the treatment of an inflammatory disease, as well as to a method for treating an inflammatory disease, said method comprising administering a modified interfering RNA or a pharmaceutical composition to a patient in need thereof.

The inflammatory disease can be a rheumatic disease and/or a connective tissue diseases, such as rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjorgren's syndrome, inflammatory bowel disease, and Crohn's disease.

The inflammatory disease can also be a non-rheumatic inflammation, like bursitis, synovitis, capsulitis, tendinitis and/or other inflammatory lesions of traumatic and/or sportive origin.

The modified interfering RNAs disclosed herein can be utilized for as research reagents for diagnostics, therapeutics and prophylaxis. In research, the modified interfering RNA can be used to specifically inhibit the synthesis of target genes in cells and experimental animals thereby facilitating functional analysis of the target or an appraisal of its usefulness as a target for therapeutic intervention. In diagnostics, the modified interfering RNA can be used to detect and quantitate target expression in cell and tissues by Northern blotting, in-situ hybridisation or similar techniques. For therapeutics, an animal or a human, suspected of having a disease or disorder, which modulating the expression of target can treat is treated by administering the modified interfering RNA compounds as detailed herein. Further provided are methods of treating an animal, particularly a mouse and rat, and methods of treating a human, suspected of having or being prone to a disease or condition, associated with expression of target by administering a therapeutically or prophylactically effective amount of one or more of the modified interfering RNA compounds or compositions disclosed herein.

### D. KITS

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for reducing or completely abrogating the off-target response to interfering RNA, the kit comprising one or more reagent compositions and one or more components or reagents for capture of the target nucleic acid, tHDA amplification, detection of amplification products, or both.

For example, the kits can comprise one or more compounds of Formula I:

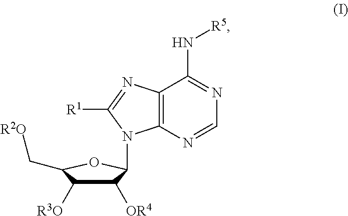

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, optionally substituted aryl, or (viii) substituted heteroaryl; and wherein R² can be a protecting group; and wherein R⁴ can be a protecting group; and wherein R⁵ can be a protecting group.

Also disclosed are kits comprising one or more compounds of Formula I, wherein R³ can be a protected phosphate. In an aspect, R¹ can be OR⁶ and wherein R⁶ can be:

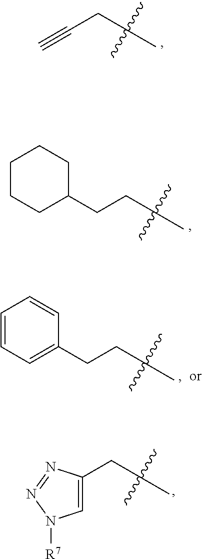

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vii) substituted C1-C12 heteroalkynyl, (viii) substituted aryl, or (ix) substituted heteroaryl. In an aspect. R⁷ can be:

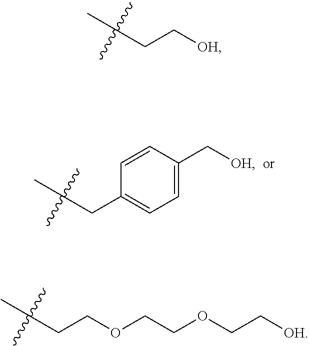

Also disclosed herein are kits comprising at least one nucleoside of Formula II:

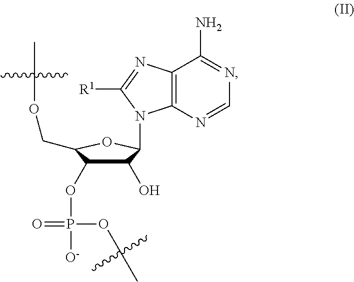

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, (viii) substituted aryl, or (ix) substituted heteroaryl. In an aspect, R¹ can be chloride. In an aspect, R¹ can be OR⁶; wherein R⁶ can be:

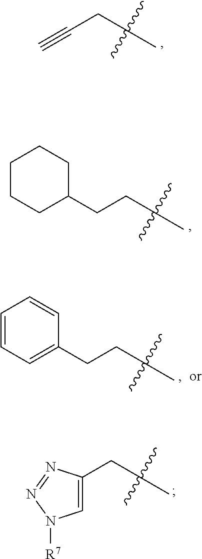

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (ix) substituted heteroaryl. In an aspect, wherein R⁷ can be:

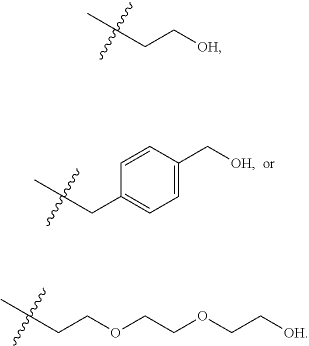

Also disclosed herein are kits comprising at least one nucleoside represented by the formula:

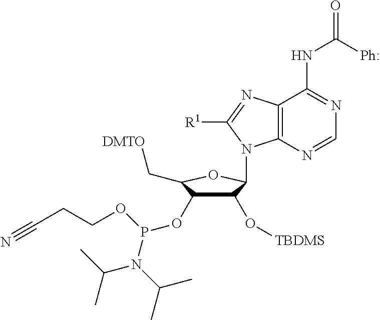

Also disclosed herein are kits comprising at least one oligonucleotide of Formula II:

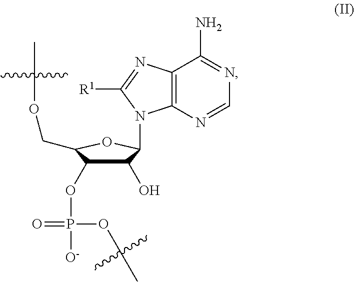

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, (viii) substituted aryl, or (ix) substituted heteroaryl. In as aspect, R⁶ of the disclosed oligonucleotide can be:

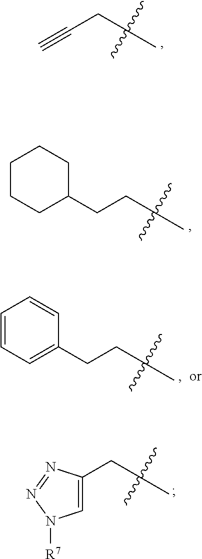

and
R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (viii) substituted heteroaryl. In as aspect, R⁷ can be:

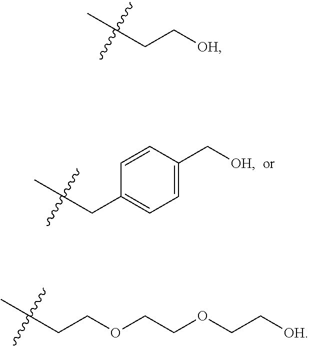

Also disclosed herein are kits comprising at least one polynucleotide of Formula II:

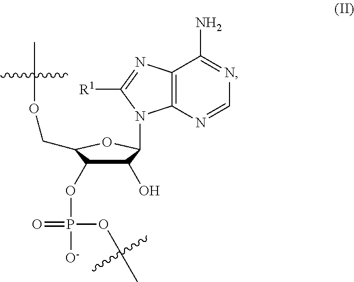

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C1-C12 alkenyl, (iv) substituted C1-C12 heteroalkenyl, (v) substituted C1-C12 alkynyl, (vi) substituted C1-C12 heteroalkynyl, (vii) substituted aryl, or (viii) substituted heteroaryl. In as aspect, R⁷ can be:

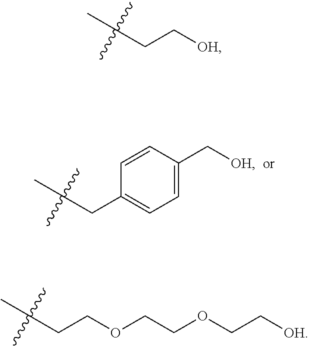

Also disclosed herein are sets of nucleotides comprising compounds of Formula I, wherein R¹ can be halide or OR⁶; wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, optionally substituted aryl, or (viii) substituted heteroaryl; and wherein R² can be a protecting group; and wherein R⁴ can be a protecting group; and wherein R⁵ can be a protecting group. In an aspect, R³ can be a protected phosphate. In an aspect, R¹ can be OR⁶ and R⁶ can be:

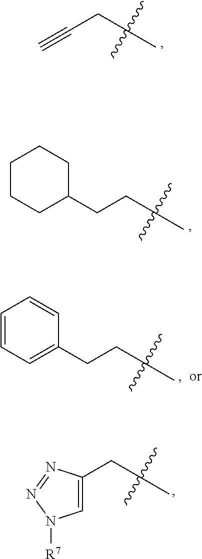

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vii) substituted C1-C12 heteroalkynyl, (viii) substituted aryl, or (ix) substituted heteroaryl. In an aspect, R⁷ can be:

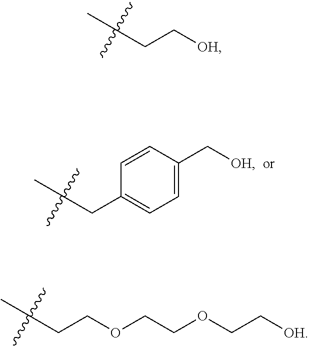

Also disclosed herein are sets of nucleosides comprising compounds of Formula II, wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C12 alkynyl, (vii) substituted C1-C12 heteroalkynyl, (viii) substituted aryl, or (ix) substituted heteroaryl. In an aspect, R¹ can be chloride. In an aspect, R¹ can be OR⁶ and R⁶ can be:

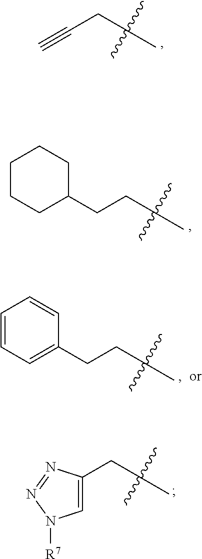

and
wherein R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (ix) substituted heteroaryl. In an aspect, R⁷ can be:

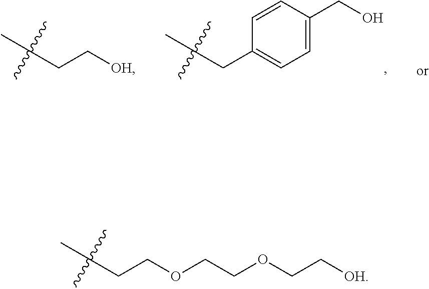

Disclosed herein is a nucleobase represented by the formula:

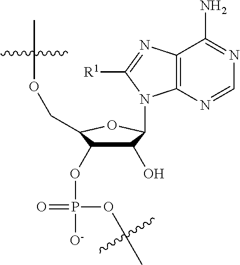

wherein R¹ can be halide or OR⁶; and wherein R⁶ can be (i) substituted C1-C12 alkyl, (ii) substituted C1-C12 heteroalkyl, (iii) substituted C1-C12 cyclo- or heterocycloalkyl, (iv) substituted C1-C12 alkenyl, (v) substituted C1-C12 heteroalkenyl, (vi) substituted C1-C₁₂ alkynyl, (vii) substituted C1-C12 heteroalkynyl, (viii) substituted aryl, or (ix) substituted heteroaryl. In as aspect, R⁶ of the disclosed polynucleotide can be:

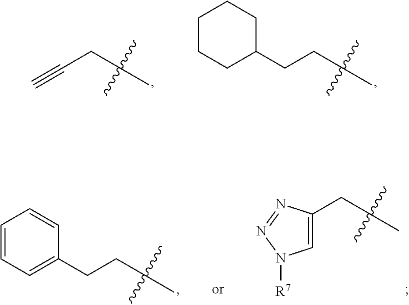

and
R⁷ can be (i) substituted C1-C12 alkyl, (ii) substituted C1-C12 heteroalkyl, (iii) substituted C1-C12 alkenyl, (iv) substituted C1-C12 heteroalkenyl, (v) substituted C1-C12 alkynyl, (vi) substituted C1-C12 heteroalkynyl, (vii) substituted aryl, or (viii) substituted heteroaryl. In as aspect, R⁷ can be:

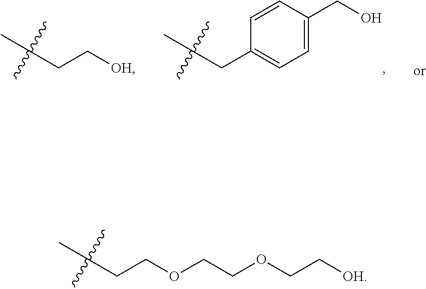

Also disclosed is a nucleic acid strand comprising at least one residue represented by the Formula I:

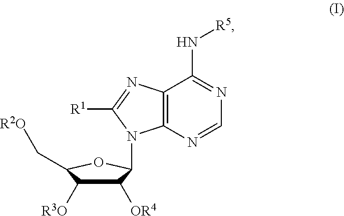

wherein R1 can be halide or OR6; and wherein R⁶ can be substituted C1-C12 alkyl, substituted C1-C12 heteroalkyl, substituted C1-C12 cyclo- or heterocycloalkyl, substituted C1-C12 alkenyl, substituted C1-C12 heteroalkenyl, substituted C1-C12 alkynyl, substituted C1-C12 heteroalkynyl, substituted aryl, or substituted heteroaryl; and wherein R2 comprises a protecting group; and wherein R4 comprises a protecting group; and wherein R5 comprises a protecting group. In an aspect, wherein R3 can be a protected phosphate. In an aspect, the nucleic acid strand comprises one or more residues of Formula I.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different RNAs and nucleobases are disclosed and discussed, each and every combination and permutation of the RNA and nucleobase are specifically contemplated unless specifically indicated to the contrary. Thus, if a class of compounds A, B, and C are disclosed as well as a class of compounds D, E, and F and an example of a combination compound, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### E. MIXTURES

Disclosed are mixtures formed by preparing the disclosed composition or performing or preparing to perform the disclosed methods. Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### F. SYSTEMS

Disclosed are systems useful for performing, or aiding in the performance of, the disclosed methods. Also disclosed are systems for producing reagent compositions. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising solid supports and reagent compositions.

### G. DATA STRUCTURES AND COMPUTER CONTROL

Disclosed are data structures used in, generated by, or generated from, the disclosed method. Data structures generally are any form of data, information, and/or objects collected, organized, stored, and/or embodied in a composition or medium. A target fingerprint stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

The method disclosed herein can, in part, be implemented via a general-purpose computing device in the form of a computer. The components of the computer can include, but are not limited to, one or more processors or processing units, a system memory, and a system bus that couples various system components including the processor to the system memory.

The system bus represents one or more of several possible types of bus structures, including a memory bus or memory controller, a peripheral bus, an accelerated graphics port, and a processor or local bus using any of a variety of bus architectures. By way of example, such architectures can include an Industry Standard Architecture (USA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. This bus, and all buses specified in this description can also be implemented over a wired or wireless network connection. The bus, and all buses specified in this description can also be implemented over a wired or wireless network connection and each of the subsystems, including the processor, a mass storage device, an operating system, software, data, a network adapter, system memory, an Input/Output Interface, a display adapter, a display device, and a human machine interface, can be contained within one or more remote computing devices at physically separate locations, connected through buses of this form, in effect implementing a fully distributed system.

The computer typically includes a variety of computer readable media. Such media can be any available media that is accessible by the computer and includes both volatile and non-volatile media, removable and non-removable media. The system memory includes computer readable media in the form of volatile memory, such as random access memory (RAM), and/or non-volatile memory, such as read only memory (ROM). The system memory typically contains data such as melting temperature data or steric data and/or program modules such as operating system and melting temperature or steric software that are immediately accessible to and/or are presently operated on by the processing unit.

The computer may also include other removable/non-removable, volatile/non-volatile computer storage media. By way of example, a mass storage device can provide non-volatile storage of computer code, computer readable instructions, data structures, program modules, and other data for the computer. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored on the mass storage device, including by way of example, an operating system and software. Each of the operating system and software (or some combination thereof) may include elements of the programming and the software. Data can also be stored on the mass storage device and can be stored in any of one or more databases known in the art. Examples of such databases include, DB2®, Microsoft® Access, Microsoft® SQL Server, Oracle®, mySQL, PostgreSQL, and the like. The databases can be centralized or distributed across multiple systems, such as across multiple laboratories or facilities.

A user can enter commands and information into the computer via an input device. Examples of such input devices include, but are not limited to, a keyboard, pointing device (e.g., a “mouse”), a microphone, a joystick, a serial port, a scanner, and the like. These and other input devices can be connected to the processing unit via a human machine interface that is coupled to the system bus, but may be connected by other interface and bus structures, such as a parallel port, game port, or a universal serial bus (USB).

A display device can also be connected to the system bus via an interface, such as a display adapter. A computer can have more than one display adapter and a computer can have more than one display device. For example, a display device can be a monitor, an LCD (Liquid Crystal Display), or a projector. In addition to the display device, other output peripheral devices can include components such as speakers and a printer which can be connected to the computer via Input/Output Interface.

The computer can operate in a networked environment using logical connections to one or more remote computing devices. By way of example, a remote computing device can be a personal computer, portable computer, a server, a router, a network computer, a peer device or other common network node, and so on. Logical connections between the computer and a remote computing device can be made via a local area network (LAN) and a general wide area network (WAN). Such network connections can be through a network adapter. A network adapter can be implemented in both wired and wireless environments. Such networking environments are commonplace in offices, enterprise-wide computer networks, intranets, and the Internet.

An implementation of application software may be stored on or transmitted across some form of computer readable media. Computer readable media can be any available media that can be accessed by a computer. By way of example, and not limitation, computer readable media may comprise “computer storage media” and “communications media.” “Computer storage media” include volatile and non-volatile, removable and non-removable media implemented in any method or technology for storage of information such as computer readable instructions, data structures, program modules, or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Modified siRNA Molecules

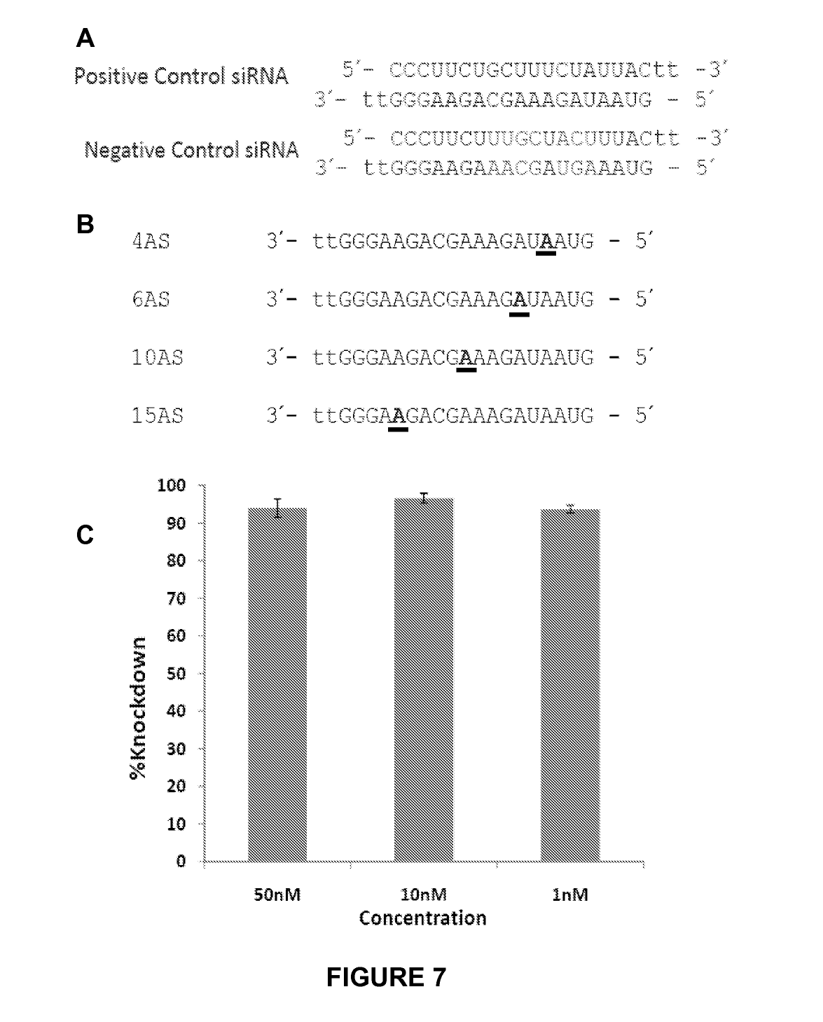

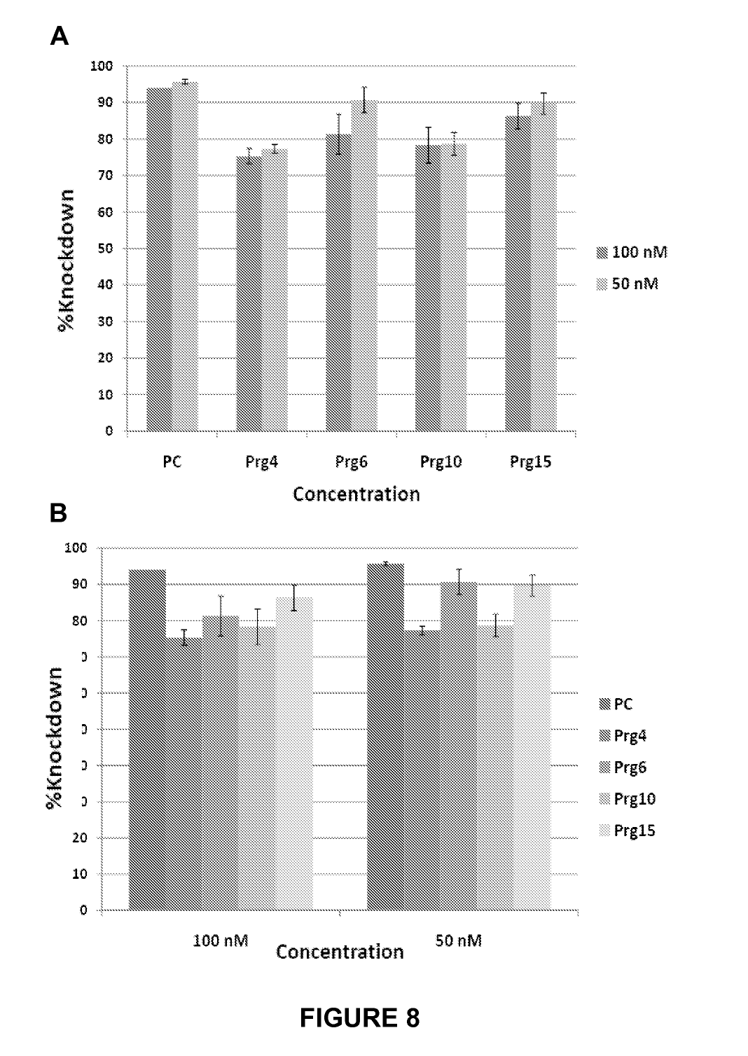

8-methoxyadenosine phosphoramidite was synthesized and incorporated into the anti-sense strand of caspase 2 siRNA. FIG. 7A shows a schematic of the double-stranded positive control siRNA (SEQ ID NO: 20 (top) and SEQ ID NO: 21 (bottom)) and the double-stranded negative control siRNA (SEQ ID NO: 22 (top) and SEQ ID NO: 23 (bottom). FIG. 7B shows a schematic for a singly modified siRNA, wherein the modification can be, but is not limited to, 8-proparglyoxyadenosine, 8-phenethyloxyadenosine, and 8-cyclohyexylethyloxyadenosine. In FIG. 7B, 4AS corresponds to SEQ ID NO: 24, 6AS corresponds to SEQ ID NO: 25, 10AS corresponds to SEQ ID NO: 26, and 15AS corresponds to SEQ ID NO: 27.

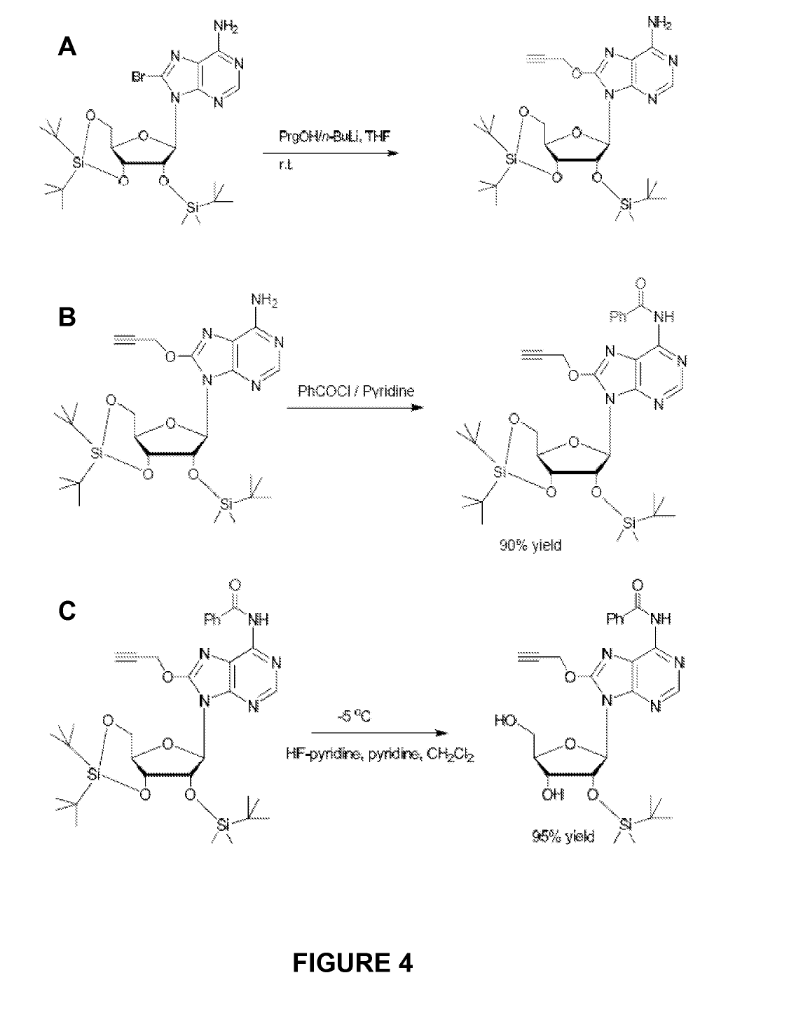

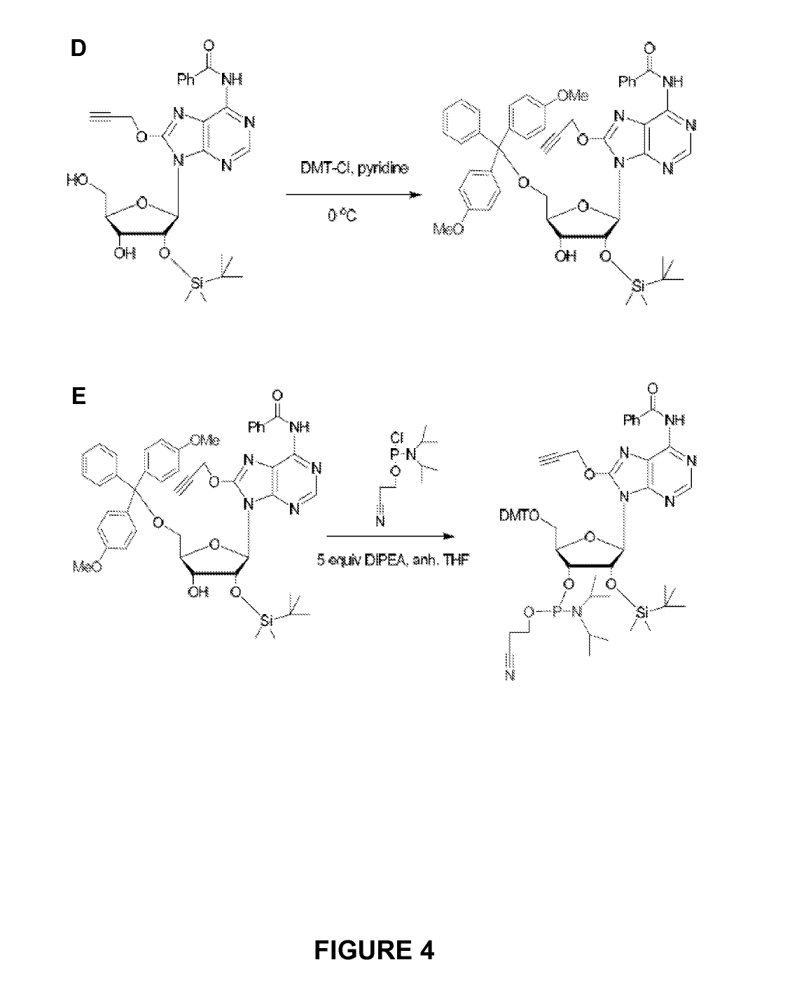

The bromination of adenosine is known in the art; generally, a three to four-fold excess of bromine generates a yield of about 75% to about 82.5% of 8-bromoadenosine. To protect the 2′-OH group during the synthesis of ribo-phosphoramidites, the 5′-OH of N⁶-benzoyladenosine was first protected; and then t-butyldimethylsilyl chloride (TBDMS-Cl) was used to protect the 2′-OH. Although the addition of Ag⁺ion is recommended to minimize unavoidable reaction at the 3′-OH, the unwanted reaction generally occurs and the overall yield is significantly reduced. Therefore, to accomplish a good overall yield, a novel protecting group di-tbutylsilyl ditriflate (DTBSDT), which protects the 5′-OH and the 3′-OH simultaneously and leaves the 2′-OH ready to be protected by TBDMS-Cl was used. The two reactions were basically a one-pot reaction, which eliminated the need for tedious separation. The reaction was almost quantitative with about 96% to about 98% product yield observed. (See FIGS. 3 and 4).

Synthesis of the methoxy derivative using sodium methoxide reagent in methanol yielded the 8-methoxyadenosine derivative as well as the 8-oxoadenosine derivative. The separation of these two derivates was not trivial and the yield of the desired compound was below 50%. Thus, the methoxy anion was generated in situ by reacting n-BuLi with excess anhydrous methanol. This reaction was much more efficient and yielded about 85% to about 93% of the desired product. The deprotection of 5′-OH and the 3′-OH was accomplished using a special fluoride reagent, HF-pyridine, at sub-zero temperature. These DMT reactions generated low yields (about 40% to about 50%) of the desired product. The phosphoramidite synthesis step yielded about 95% to about 98% of desired product. The 8-methoxyadenosine phosphoramidite was then incorporated into the antisense strand at position 9 or 14 (opposite to positions 11 and 6 of the sense strand, respectively), or both positions 9 and 14. The propargyl moiety at position 8 of adenosine can simplify the syntheses of other position 8 substituted adenosine analogs. The alkyne moiety of 8-propargyladenosine in siRNA can be “clicked” with suitable water-soluble azides leading to the formation of desirable minor groove modification.

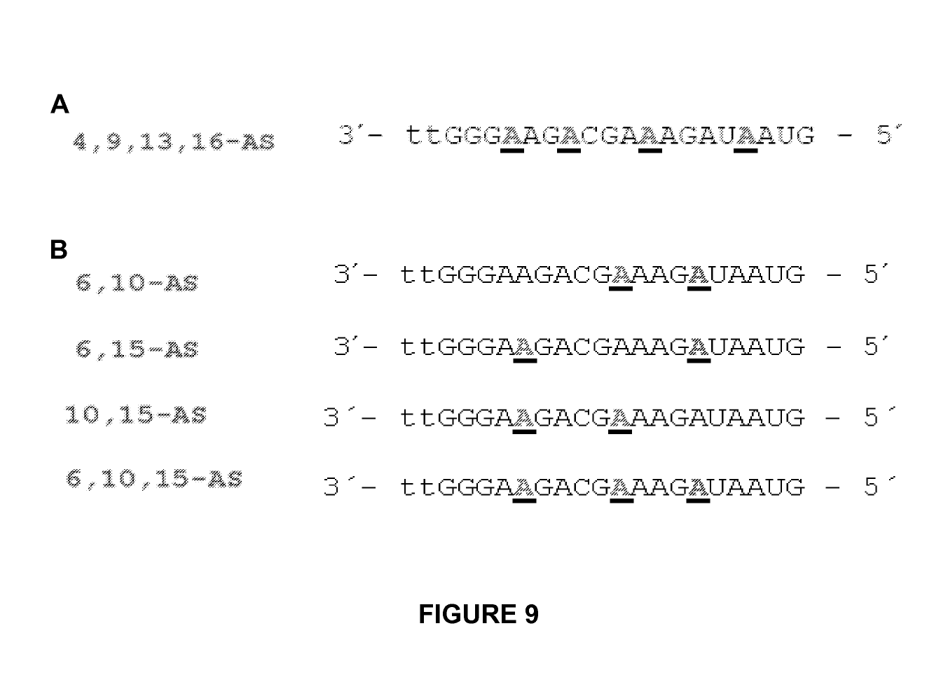

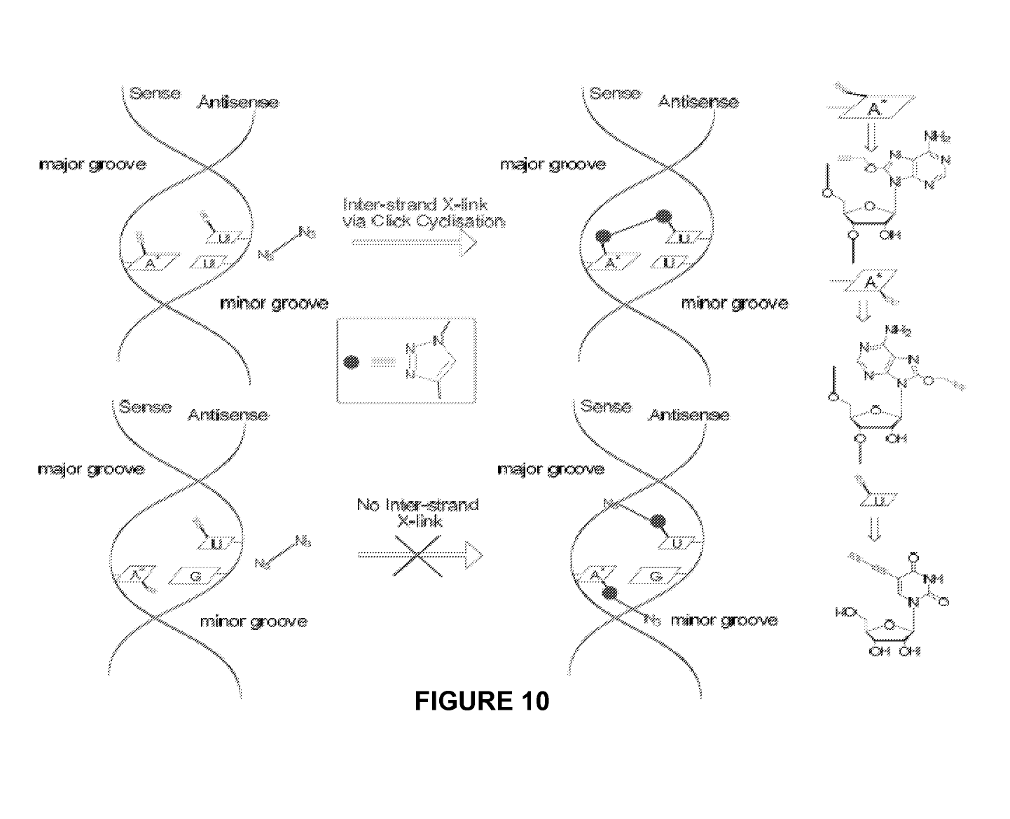

Multiple modifications were also made to the anti-sense strand of caspase 2 siRNA. FIG. 9A shows a schematic of the positive control siRNA with modifications at positions 4, 9, 13, and 16 (SEQ ID NO: 28). FIG. 9B shows a schematic of the positive control siRNA with various combinations of multiple modifications. From top to bottom, the modifications are at positions 6 and 10 (SEQ ID NO: 29), at positions 6 and 15 (SEQ ID NO: 30), at positions 10 and 15 (SEQ ID NO: 31), and at positions 6, 10, and 15 (SEQ ID NO: 32). The modifications at these positions can be, but are not limited to, 8-proparglyoxyadenosine, 8-phenethyloxyadenosine, and 8-cyclohyexylethyloxyadenosine.

### Example 2

### Effect of Base Modification on RNAi

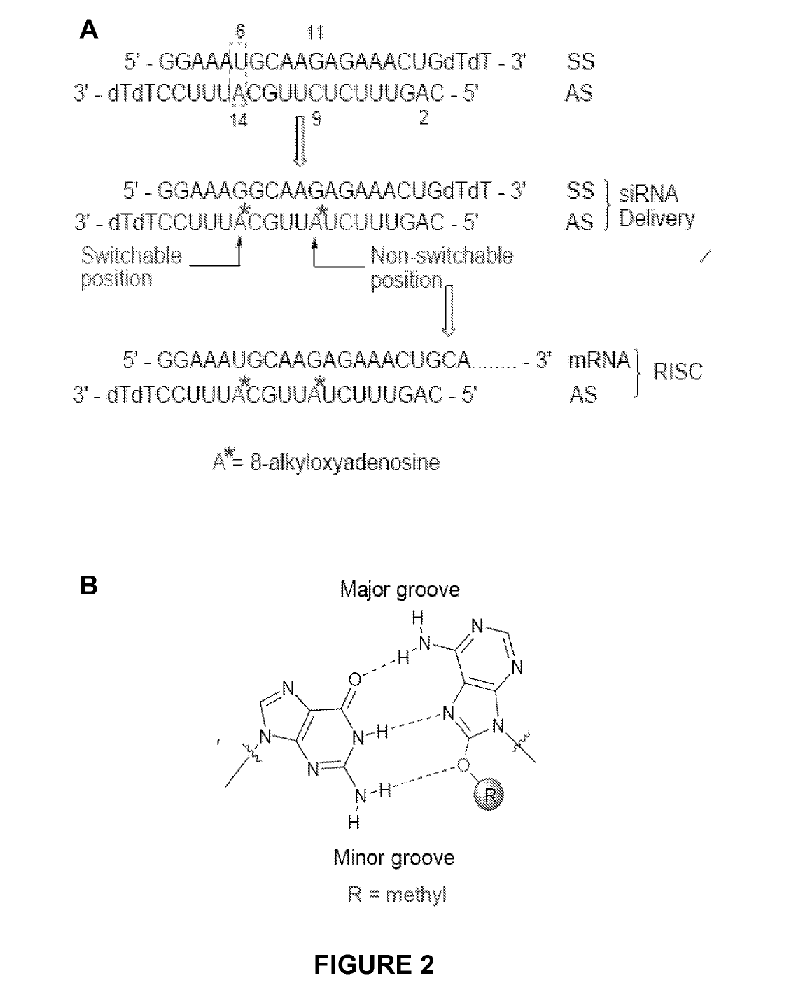

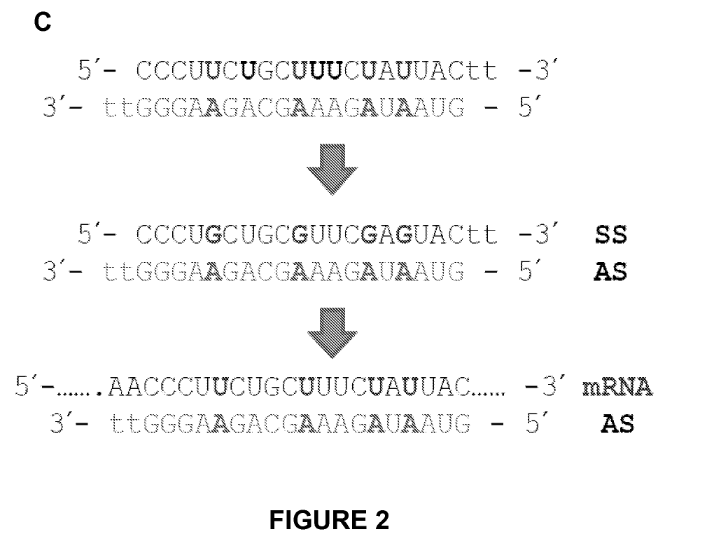

Using the standard caspase 2 siRNA, the 8-alkyloxyadenosine ‘base-switch’ was applied at two positions in the antisense strand. For example, see FIG. 2, which shows the schematic for 8-Methoxyadenosine in switchable and persistent steric crowding of the minor groove. In the top panel of FIG. 2A, the SS strand is represented by SEQ ID NO: 1 and the AS strand is represented by SEQ ID NO: 2. In the middle panel of FIG. 2A, the SS strand is represented by SEQ ID NO: 3 and the AS strand is represented by SEQ ID NO: 4. In the bottom panel of FIG. 2A, the SS strand is represented by SEQ ID NO: 5 and the AS strand is represented by SEQ ID NO: 6.

These experiments showed that the 8-alkyloxyadenosine ‘base-switch’ occurred at positions 2 and 14; however, as position 2 is near the end of the 5′-terminus, it is unlikely to play any significant role in preventing the binding to dsRBMs of cellular proteins. These studies also determined that a modified adenosine could be substituted for the C at position 9.

Three modified anti-sense strands were synthesized by the automated solid phase synthesizer. The antisense strands contained modified adenosines at either position 9, or position 14, or at both positions 9 and 14. All the siRNA oligomers were then deprotected using methanolic ammonia and triethylamine trihydrofluoride and later purified by HPLC. The position 9 substitution was designed to explore the base switching mechanism. Other experiments can utilize substitutions at positions 6, 11, and 14 in a non-switchable mode. For example, in FIG. 2C, BndG modifications at positions 6, 9, 11, and 14 of the sense strands disrupt PKR interactions. In the top panel of FIG. 2C, the 5′-3′ strand corresponds to SEQ ID NO: 7 while the 3′-5′ strand corresponds to SEQ ID NO: 8. In the middle panel of FIG. 2C, the 5′-3′ strand corresponds to SEQ ID NO: 9 while the 3′-5′ strand corresponds to SEQ ID NO: 10. In the bottom panel of FIG. 2C, the 5′-3′ strand corresponds to SEQ ID NO: 11 while the 3′-5′ strand corresponds to SEQ ID NO: 12.

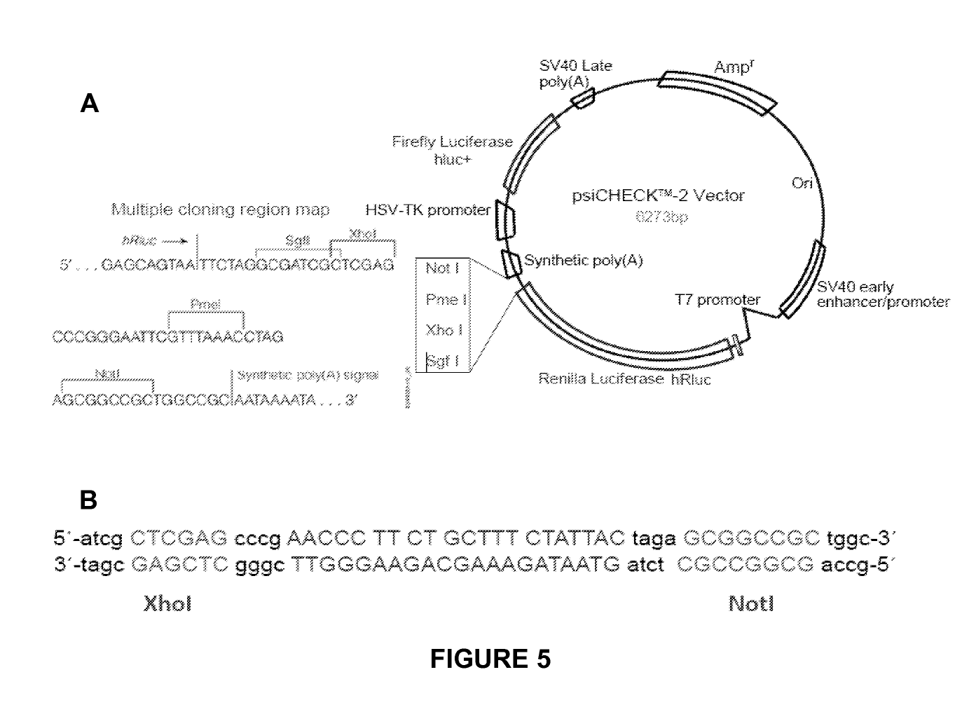

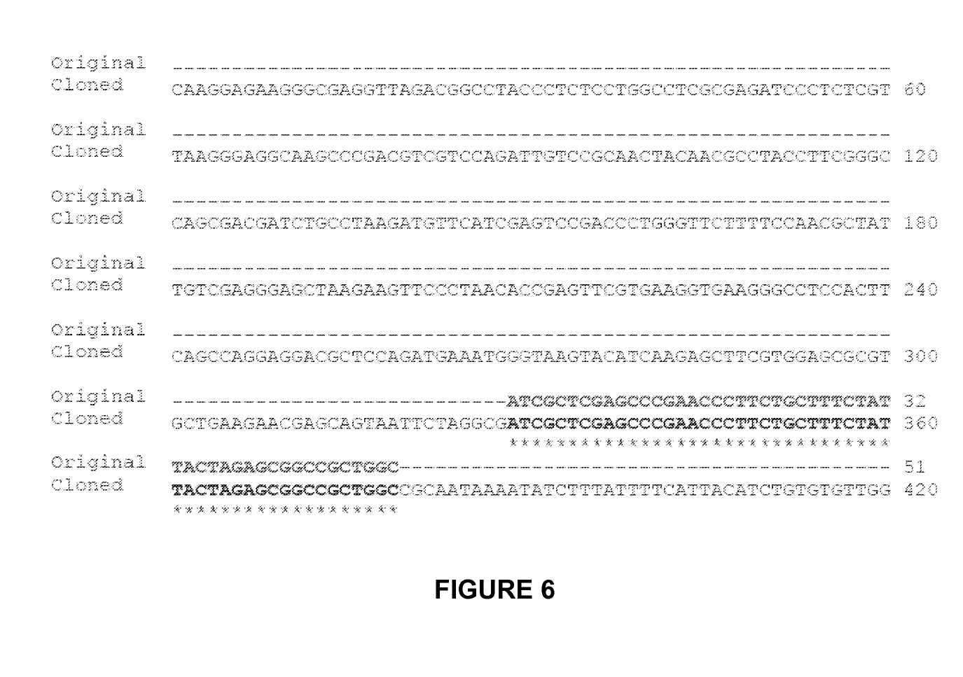

The caspase 2 knock-down study using 8-alkyloxyadenosine indicated that these ‘persistent’ minor groove blockades somewhat reduced RNAi activity. (Table 1). Modified psiCHECK2 plasmid containing two segments for the expression of dual luciferase (firefly and renilla was used) in this gene knock-down study. Modification was achieved by specific nicking of vector psiCHECK2 plasmid by Not I and Xho I restriction endonucleases and inserting a selected fragment of caspase 2 DNA fragment. FIG. 5 shows the schematic for psiCHECK2 vector. FIG. 5A shows the sequences corresponding to SEQ ID NO: 13, SEQ ID NO: 14, and SEQ ID NO: 15. In FIG. 5B, the top sequence reading in the 5′ to 3′ direction corresponds to SEQ ID NO: 16. The bottom sequence reading in the 3′ to 5′ direction corresponds to SEQ ID NO: 17. FIG. 6 shows the sequence of the original plasmid (top sequence; SEQ ID NO: 18) compared to the sequence of the recombinant plasmid (bottom sequence; SEQ ID NO: 19).

The caspase 2 inserted plasmid can be redesigned by arranging some of the bases so that switchable adenosine analogs can be placed at one or more of positions 6, 9, 11, and 14 positions. HeLa cells were transfected normally with plasmid and siRNA for the knock-down studies. Positive control contained plasmid only. The modification at position 14 cleaved the mRNA. Significant knockdown was recorded at concentration as low as 10 nM. Table 2 shows knock-down of caspase 2 using various concentrations of 8-methoyxadenosine. These data indicate that the siRNA activity is observed at very low concentrations of siRNA comprising the modified adensones.

TABLE 1
Caspase 2 Knockdown Studies using
8-alkyloxyadenosine Modifications

| Modifications | % Knockdown | % Knockdown | % Knockdown |
|---|---|---|---|
| (Positions) | (1000 nM) | (100 nM) | (10 nM) |
| 9 | −11.8 | −33.6 | −11.7 |
| 14 | 19.0 | 52.8 | 47.6 |
| 9 and 14 | 16.3 | −3.1 | −6.1 |
| Unmodified | — | 76.6 | 80.4 |
| Positive Control | N.A. | 0 | N.A |

TABLE 2
Caspase 2 Knockdown Studies using
8-methoxyadenosine Modifications

| Modifications | % Knockdown | | | | | |
|---|---|---|---|---|---|---|
| (Positions) | 100 nM | 10 nM | 1 nM | 100 pM | 10 pM | 1 pM |
| 9-OMe | 13 | 53 | 50 | 54 | 55 | 62 |
| 14-OMe | 81 | 83 | 73 | 53 | 43 | 52 |
| Positive Control | 89 | 85 | 76 | 57 | 20 | 48 |

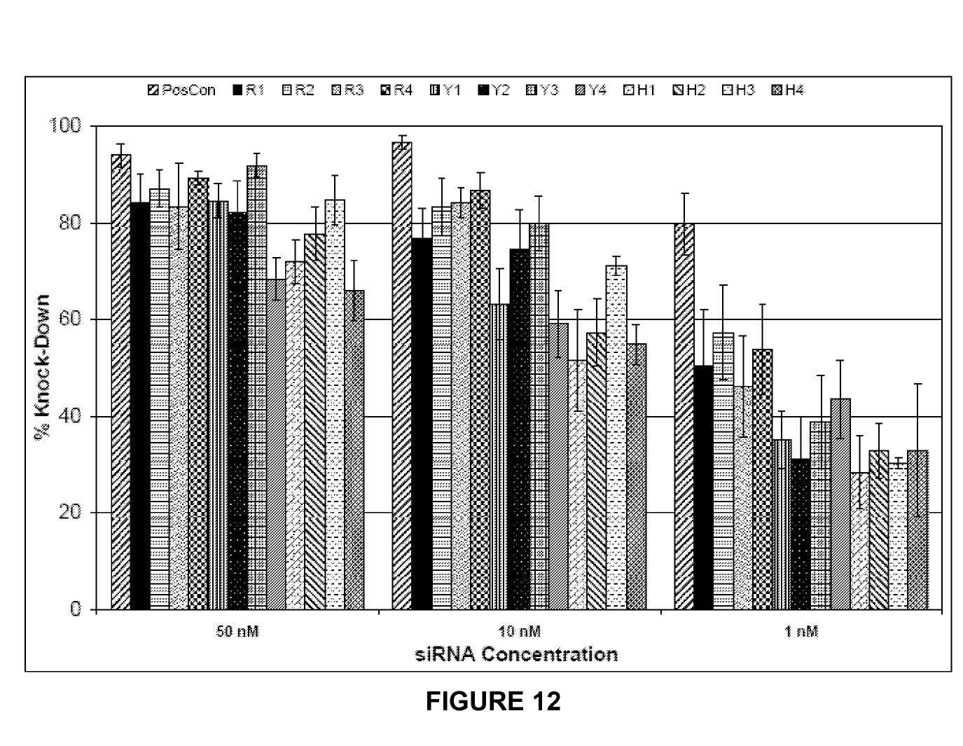

Also, FIG. 12 shows the use of singly modified siRNA to knock down caspase 2. In FIG. 12, the following apply: PosCon is a positive control siRNA; R1 is a propargyloxy modification at position 4; R2 is a propargyloxy modification at position 6; R3 is a propargyloxy modification at position 10; R4 is a propargyloxy modification at position 15; Y1 is a cyclohexylethyloxy modification at position 4; Y2 is a cyclohexylethyloxy modification at position 6; Y3 is a cyclohexylethyloxy modification at position 10; Y4 is a cyclohexylethyloxy modification at position 15; H1 is a phenethyloxy modification at position 4; H2 is a phenethyloxy modification at position 6; H3 is a phenethyloxy modification at position 10; and H4 is a phenethyloxy modification at position 15.

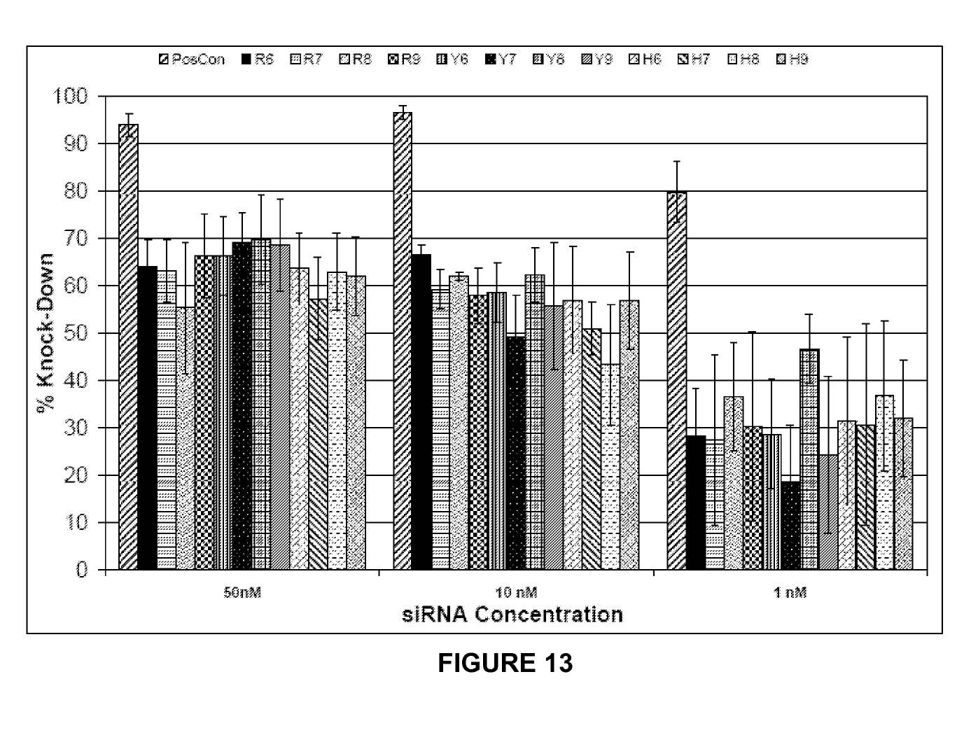

FIG. 13 shows the use of multiply modified siRNA to knock down caspase 2. In FIG. 13, the following apply: PosCon is a positive control siRNA; R6 is a propargyloxy modification at positions 6 and 10; R7 is a propargyloxy modification at positions 6 and 15; R8 is a propargyloxy modification at positions 10 and 15; R9 is a propargyloxy modification at positions 6, 10, and 15; Y6 is a cyclohexylethyloxy modification at positions 6 and 10; Y7 is a cyclohexylethyloxy modification at positions 6 and 15; Y8 is a cyclohexylethyloxy modification at positions 10 and 15; Y9 is a cyclohexylethyloxy modification at positions 6, 10, and 15; H6 is a phenethyloxy modification at positions 6 and 10; H7 is a phenethyloxy modification at positions 6 and 15; H8 is a phenethyloxy modification at positions 10 and 15; and H9 is a phenethyloxy modification at positions 6, 10, and 15.

### Example 3

### T_{M} Analysis of Modified and Unmodified Adenosines

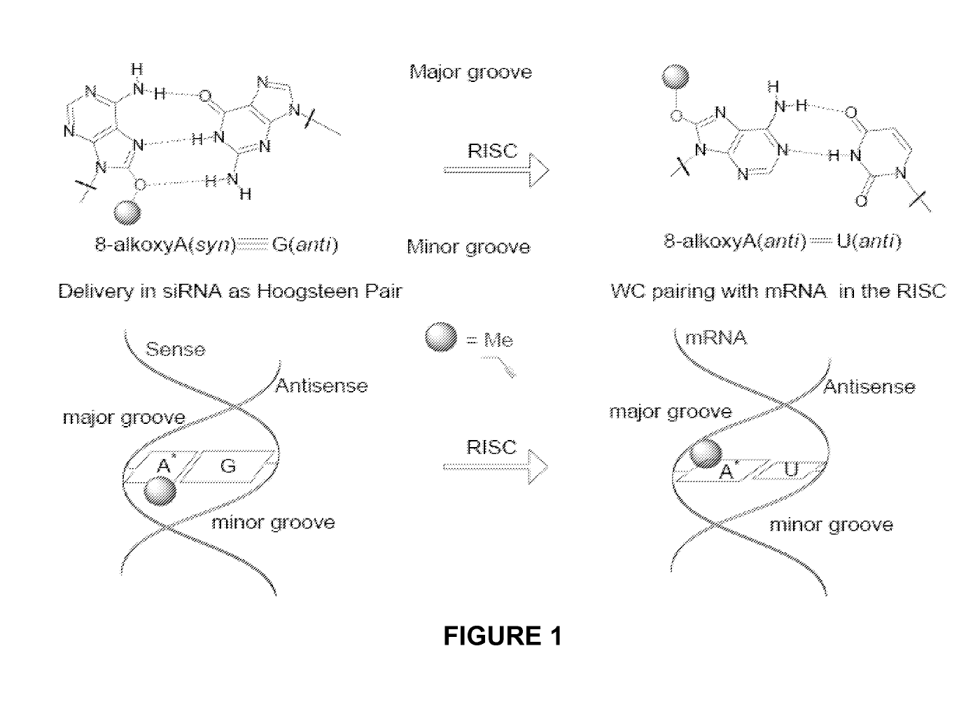

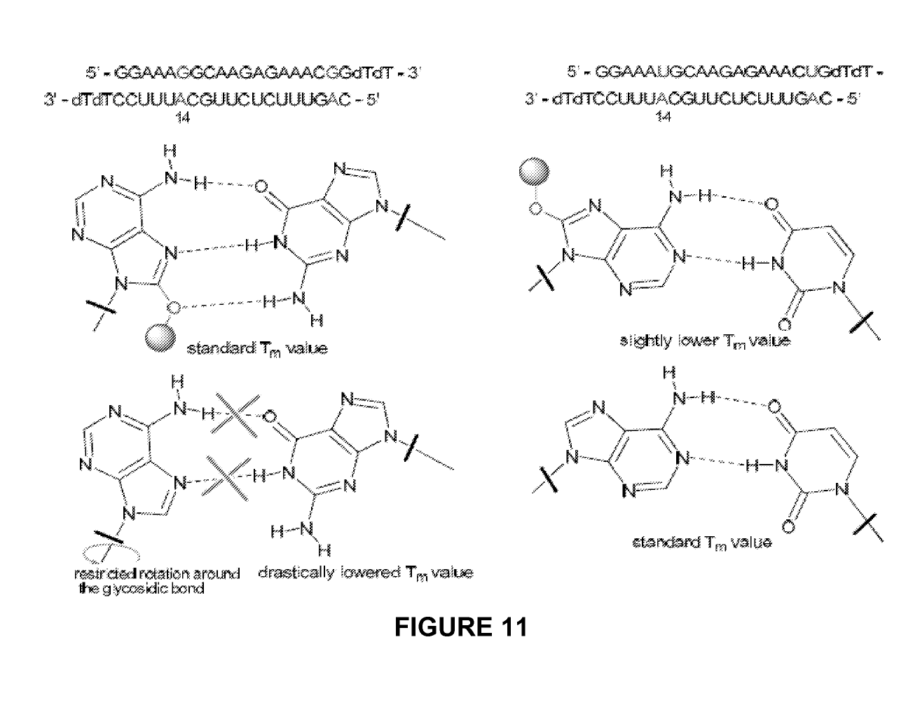

The melting temperature (T_{M}) analysis of the siRNA oligomers containing modified and unmodified adenosines when placed opposite to G or U can be used to examine base switching. (See FIG. 1). Furthermore, FIG. 11 shows a schematic for using T_{M} analysis to analyze base switching and click chemistry. On the top hand side of the figure, the 5′-3′ siRNA corresponds to SEQ ID NO: 33, while the 3′-5′ siRNA corresponds to SEQ ID NO: 34. On the top right hand side of the figure, the 5′-3′ siRNA corresponds to SEQ ID NO: 35, while the 3′-5′ siRNA corresponds to SEQ ID NO: 360.

Modified nucleosides have a natural inclination to adopt syn conformation around the glycosidic bond. Thus, during delivery, the modified base will pair with G by exposing its Hoogsteen face and thereby projecting its steric bulk into the minor groove. In the RISC, when the AS complements with a suitable mRNA, the modified adenosine base will encounter U opposite to it. This U will force the modified nucleoside to flip to its anti conformation where the steric bulk will reside in the deep major groove.

Depending on the syn or anti conformation around the glycosidic bond, 8-methoxyadenosine can base pair with either G or U. Because of a strong preference for A in the anti conformation, 8-methoxyadenosine can form H-bonding with U; however, H-bonding with G requires syn orientation of A. Thus, because 8-methoxyadenosine:U has the bulky methoxy group, the T_{M} of the siRNA duplex with A:U or 8-methoxyadenosine:U base pairs can modestly differ. The T_{M} of duplex dsRNAs with 8-methoxyadenosine:G and A:G base pairs can significantly differ.

TABLE 3
T_{M} Analysis of Singly Modified Antisense Strands

| Duplexes | Tm 8-ROA:G | Tm 8-ROA:U |
|---|---|---|
| Unmodified A:U | | 65.7 (0.6) |
| Propargyl 6 | 62.6 (0.4) | 63.1 (0.4) |
| Propargyl 10 | 60.3 (0.7) | 63.2 (0.7) |
| Propargyl 15 | 58.2 (0.6) | 63.8 (0.4) |
| Phenethyl 6 | 62.3 (0.6) | 62.6 (0.6) |
| Phenethyl 10 | 59.2 (0.6) | 60.8 (0.5) |
| Phenethyl 15 | 57.1 (0.4) | 59.8 (0.7) |
| Cyclohexylethyl 6 | 62 (0.4) | 62.3 (0.7) |
| Cyclohexylethyl 10 | 60 (0.7) | 62.7 (1.0) |
| Cyclohexylethyl 15 | 57.5 (0.5) | 59.9 (0.6) |
| Mismatch 6(A:G) | 54.3 (0.6) | |
| Mismatch 10(A:G) | 53.2 (0.4) | |
| Mismatch 15(A:G) | 54.4 (0.3) | |

“Click” chemistry can also be conveniently used to investigate base switching. (See FIGS. 1 and 11). An siRNA duplex can be constructed in which 8-propargyloxyA is placed opposite to G so that the alkynyl moiety will be projected in the minor groove. An siRNA duplex can be constructed in which 8-propargyloxyA is placed opposite to U, so that the alkynyl moiety will be projected in the major groove. Another alkynyl modified nucleoside can be placed just above G or just below G. In this arrangement, the two alkynyl groups from two strands can be projected in the minor groove. In another siRNA duplex, two alkynyl moieties can be projected into the major groove by choosing U opposite to 8-propargyloxyA and placing a 5-alkynylU just above or just below the former U. Those siRNA duplexes bearing two “clickable” moieties, both in either minor groove or major groove, can be cross-linked by using a suitable bis-azide. The projection of two alkynyl handles in two different grooves cannot lead to inter-strand covalent tethering.

In these experiments, the choice of the water-soluble azide linker can be an important decision. PEGylated bis-azides of varying length can be used. Bis-azides can be purchased from commercially available sources or can be synthesized from corresponding PEG oligomers. The triazole moiety can be bio-compatible and a hydrophilic aromatic azide and PEG-based azides can be used.

Optimization of the linker length can be utilized to prevent undesired cross-linking between minor and major groove alkyne handles. For example, if a linker is too long, then it can lead to cross-linking of two siRNA duplexes. Optimization can begin by using the shortest possible linker. Shorter bis-azides can pose less significant chance of cross-link formation between two siRNA duplexes due to formidable electrostatic repulsion between the phosphste backbones. Once one side of the bis-azide linker is “clicked” with a major-groove- or minor-groove-residing alkyne, the other azide group can find the local concentration of the second alkyne (in the same groove) so high (as compared to neighboring alkyne moieties) that it can inevitably cross-link that alkyne. Even from thermodynamic perspective, inter-strand cross-linking in a duplex (similar to intra-molecular reaction) can be preferred to inter-duplex crosslinking since the former can lead to lesser loss of entropy.

Click reaction products can be easily analyzed both by denaturing gel electrophoresis and denaturing HPLC. Under these conditions, unreacted or uncross-linked siRNA oligomers can have distinctively different behavior compared to cross-linked siRNA duplexes. Characterization of the products can be accomplished by ESI-MS or MALDI-TOF.

### Example 4

### PKR Binding Study of the Modified siRNAs

The introduction of minor groove modifications at one or more specific positions the anti-sense strand can effectively block PKR binding. The modified siRNAs can prevent PKR from using dsRBMs to wrap onto its minor grooves, thereby preventing sequence-independent off-target effects, and improving RNAi efficacy. First, PKR can be purified and the binding study with modified siRNAs can be carried out. Unmodified strands can be used as a control to measure the efficacy in blocking PKR. Then binding studies with other dsRBM containing proteins (e.g., ADAR2) can be performed.

### Example 5

### Using Electrostatic Repulsion to Prevent PKR Binding

Crystal structure of a dsRNA-protein complex can be a starting point for the examining electrostatic repulsion. The crystal structure of a dsRNA:protein complex implies that PKR, in addition to many other amino acid side chains, can utilize glutamate and histidine side chains to form H-bonds with 2′-OH of the ribose sugar. The availability of 2′-OH as a H-bonding partner at certain positions of the siRNA duplex can be important. The placement of a carboxylic acid containing moiety (of appropriate length) in the minor groove can lead to strong electrostatic repulsion between this carboxylate moiety and any incoming carboxylate groups from the glutamate side chain of PKR. Additionally, the pendent carboxylate can function as an H-bonding partner of the 2′-OH and exclude any other H-bonding partner from PKR. This steric-cum-electrostatic base switch can prohibit PKR binding more efficiently than simple steric-based probe.

PEGylated carboxylic acid can be “clicked” with the propargyloxy moiety of the 8-modified adenosine in the anti-sense strand at one or more positions. PKR, like many other dsRBM-containing proteins, binds RNAs onto their minor groves; while doing so, they also secure a firm grip on the intervening major groove. Electrostatic attraction between the side chains of several lysines and phosphate backbone can provide help to PKR during binding with two consecutive minor grooves. Major groove modifications that introduce cationic moieties in the groove thereby repelling amino group containing side chains of lysines can be designed and lysine (PKR)-phosphate backbone (dsRNA) interaction in the major groove can be nullified or can be diminished. Click chemistry can be used to connect a suitable PEG-based terminal amine with the propargyl ‘handle’ present in 8-propargyloxyadenosine nucleoside of the anti-sense strand. This major groove modification can be used in conjunction with other minor groove modifications.

### Example 6

### Synthesis of 8-Chloro-2-alkylaminoadenosine

An alternative nucleoside analog that has switching potential can also be utilized. Novel 8-chloro-2-alkylaminoadenosine analogs can be synthesized from 2-aminoadenosine, as the starting material, using the synthetic scheme described herein. The chlorination at position 8 can be optimized according to methods known in the art. During the synthesis process, a modified Mitsunobu reaction can be used to anchor diverse alkyl groups with the 2-amino group. The steric bulk of the ‘switch’ can then be projected both in the minor and major groove depending on the base pairing partner. Chemical and biological studies can be conducted to verify the switching hypothesis and the efficiency of the modified siRNAs. Also, utilizing the propargyl handle, many other sterically-demanding and charged groups can be placed to accomplish diverse goals described above.

The ‘switching’ potential of a Janus-faced base can be used to solve the sequence-independent off target effects associated with cellular proteins possessing dsRBMs during siRNA-mediated RNAi. 8-Methoxyadenosine phosphoramidite can be synthesized and incorporated in the anti-sense strand of caspase 2 siRNA. 8-MethoxyA, 8-propargyloxyA, and other modified adenosines can be substituted at positions 6, 9, and 11 of the anti-sense strand, which positions are known to block PKR biding. Following modifications to the anti-sense strand, RNAi efficacy and PKR binding ability can be tested. Comparative melting temperature analysis of the siRNA duplexes and selectivity in ‘click’ chemistry based on inter-strand crosslinking of siRNA duplexes can provide insight into the switch mechanism. A propargyloxy group at position 8 (an A) can be used to place steric bulks in the minor groove and can also be used to place charged moieties in both minor and major grooves. The placement of charged moieties in both minor and major grooves can provide an additional layer of protection against PKR binding onto dsRNAs. 8-chloro-2-alkylaminoadenosine can also be synthesized and can be tested for its RNAi efficacy as a switch and for its ability as an inhibitor in PKR-dsRNA binding.

What is claimed is:

1. A compound of formula (I): 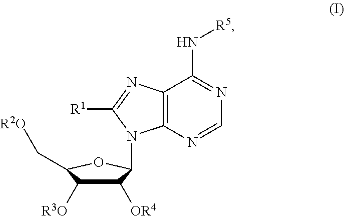
wherein R¹ is halide or OR⁶;
wherein R⁶ is optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl;
wherein R² comprises a protecting group;
wherein R⁴ comprises a protecting group; and
wherein R⁵ comprises a protecting group.

2. The compound of claim 1, wherein R³ comprises a protected phosphate.

3. The compound of claim 1, which is represented by the formula:

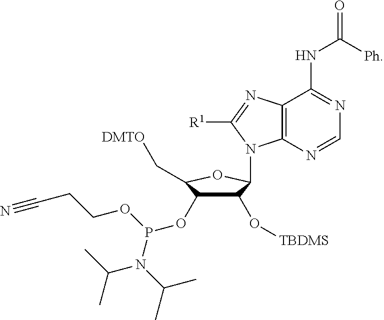

4. The compound of claim 1, wherein R¹ is chloride.

5. The compound of claim 1, wherein R¹ is OR⁶;
wherein R⁶ is: 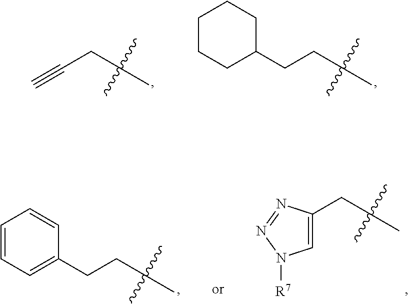
and
wherein R⁷ is optionally substituted C1-C12 alkyl, optionally substituted C1-C12 heteroalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl.

6. The compound of claim 5, wherein R⁷ is:

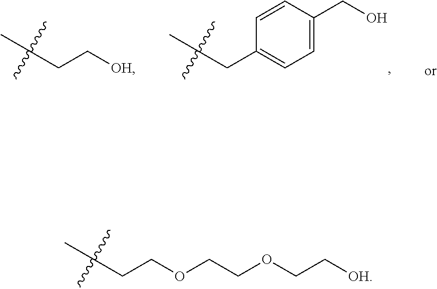

7. A nucleoside of Formula II: 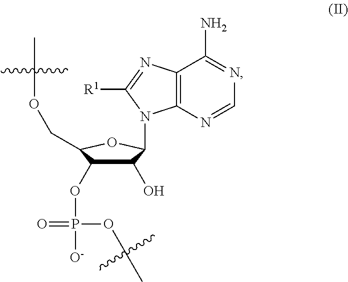
wherein R¹ is halide or OR⁶; and
wherein R⁶ is optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl.

8. The nucleoside of claim 7, wherein R¹ is chloride.

9. The nucleoside of claim 7, wherein R¹ is OR⁶;
wherein R⁶ is: 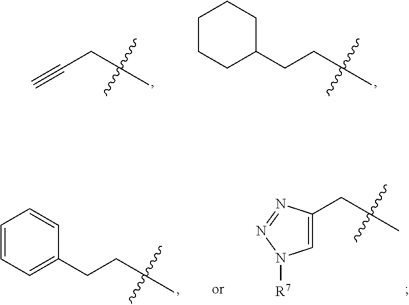
and
wherein R⁷ is optionally substituted C1-C12 alkyl, optionally substituted C1-C12 heteroalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl.

10. The nucleoside of claim 9, wherein R⁷ is:

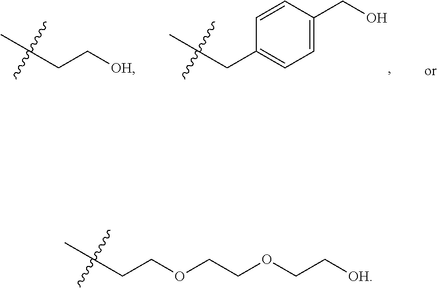

11. An oligonucleotide or polynucleotide comprising at least one nucleoside of Formula II: 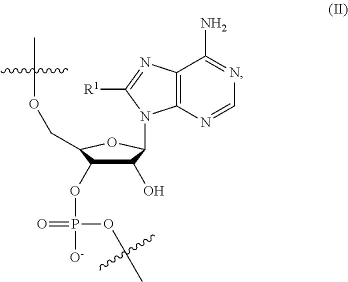
wherein R¹ is halide or OR⁶; and
wherein R⁶ is optionally substituted C1-C12 alkyl, optionally substituted C1-C12 heteroalkyl, optionally substituted C1-C12 cyclo- or heterocycloalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl.

12. The oligonucleotide or polynucleotide of claim 11, wherein R⁶ is: 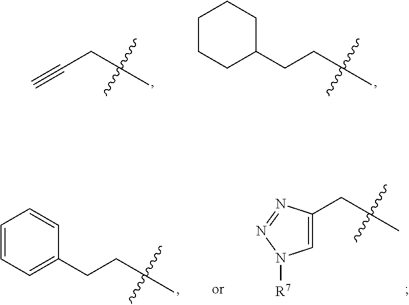
and
wherein R⁷ is optionally substituted C1-C12 alkyl, optionally substituted C1-C12 heteroalkyl, optionally substituted C1-C12 alkenyl, optionally substituted C1-C12 heteroalkenyl, optionally substituted C1-C12 alkynyl, optionally substituted C1-C12 heteroalkynyl, optionally substituted aryl, or optionally substituted heteroaryl.

13. The nucleoside of claim 12, wherein R⁷ is:

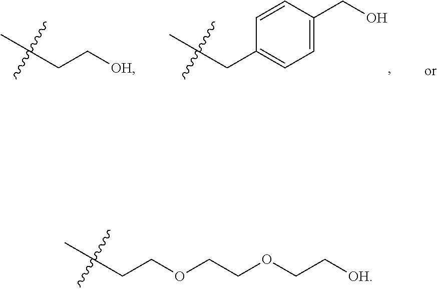

14. A method of blocking the binding of an off-target molecule to an siRNA molecule, comprising,
modifying at least one adenosine base of the siRNA molecule, and
administering to a subject the siRNA molecule.

15. The method of claim 14, wherein the siRNA molecule comprises two or more modified adenosine bases.

16. The method of claim 14, wherein the siRNA molecule comprises three or more modified adenosine bases.

17. The method of claim 14, wherein the modified adenosine base comprises the compound of claim 1.

18. The method of claim 14, wherein the off-target molecule is a double stranded RNA-binding motif (dsRBM).

19. The method of claim 18, wherein the dsRBM is RNA dependent protein kinase (PKR).

20. The method of claim 18, wherein the dsRBM is adenosine deaminase (ADAR).

21. The method of claim 14, wherein the off-target molecule is Toll-Like Receptor-7, Toll-Like Receptor-8, or Toll-Like Receptor-9.

22. The method of claim 14, wherein the efficacy of the siRNA molecule is increased.

23. An siRNA molecule comprising at least one modified adenosine.

24. The siRNA molecule of claim 23, wherein the base opposite the modified adensosine is not complementary.

25. A kit comprising the compound of claim 1.

26. A kit comprising the compound of claim 6.

27. A kit comprising the nucleoside of claim 7.

28. A kit comprising the oligonucleotide of claim 11.

29. A kit comprising the polynucleotide of claim 11.

30. A set of nucleotides comprising at least one compound of claim 1.

31. A set of nucleotides comprising at least one oligonucleotide or polynucleotide of claim 11.


SEQUENCE LISTING (36 sequences)

SEQ ID NO: 1 - DNA, 23 bp, Artificial Sequence
  Description of Artificial Sequence note = synthetic construct
ggaaaugcaagagaaacugdtdt

SEQ ID NO: 2 - DNA, 23 bp, Artificial Sequence
  Description of Artificial Sequence note = synthetic construct
dtdtccuuuacguucucuuugac

SEQ ID NO: 3 - DNA, 23 bp, Artificial Sequence
  Description of Artificial Sequence note = synthetic construct
ggaaaggcaagagaaacugdtdt

SEQ ID NO: 4 - DNA, 23 bp, Artificial Sequence
  Description of Artificial Sequence note = synthetic construct
dtdtccuuuacguuaucuuugac

SEQ ID NO: 5 - DNA, 21 bp, Artificial Sequence
  Description of Artificial Sequence note = synthetic construct
ggaaaugcaagagaaacugca

SEQ ID NO: 6 - DNA, 23 bp, Artificial Sequence
  Description of Artificial Sequence note = synthetic construct
dtdtccuuuacguuaucuuugac

SEQ ID NO: 7 - DNA, 21 bp, Artificial Sequence
  Description of Artificial Sequence note = synthetic construct
cccuucugcuuucuauuactt

SEQ ID NO: 8 - DNA, 21 bp, Artificial Sequence
  Description of Artificial Sequence note = synthetic construct
ttgggaagacgaaagauaaug

SEQ ID NO: 9 - DNA, 21 bp, Artificial Sequence
  Description of Artificial Sequence note = synthetic construct
cccugcugcguucgaguactt

SEQ ID NO: 10 - DNA, 21 bp, Artificial Sequence
  Description of Artificial Sequence note = synthetic construct
ttgggaagacgaaagauaaug

SEQ ID NO: 11 - DNA, 21 bp, Artificial Sequence
  Description of Artificial Sequence note = synthetic construct
aacccuucugcuuucuauuac

SEQ ID NO: 12 - DNA, 21 bp, Artificial Sequence
  Description of Artificial Sequence note = synthetic construct
ttgggaagacgaaagauaaug

SEQ ID NO: 13 - DNA, 28 bp, Artificial Sequence
  Description of Artificial Sequence note = synthetic construct
gagcagtaattctaggcgatcgctcgag

SEQ ID NO: 14 - DNA, 23 bp, Artificial Sequence
  Description of Artificial Sequence note = synthetic construct
cccgggaattcgtttaaacctag

SEQ ID NO: 15 - DNA, 25 bp, Artificial Sequence
  Description of Artificial Sequence note = synthetic construct
agcggccgctggccgcaataaaata

SEQ ID NO: 16 - DNA, 51 bp, Artificial Sequence
  Description of Artificial Sequence note = synthetic construct
atcgctcgagcccgaacccttctgctttctattactagagcggccgctggc

SEQ ID NO: 17 - DNA, 51 bp, Artificial Sequence
  Description of Artificial Sequence note = synthetic construct
tagcgagctcgggcttgggaagacgaaagataatgatctcgccggcgaccg

SEQ ID NO: 18 - DNA, 51 bp, Artificial Sequence; identical to SEQ ID NO: 16
  Description of Artificial Sequence note = synthetic construct

SEQ ID NO: 19 - DNA, 420 bp, Artificial Sequence
  Description of Artificial Sequence note = synthetic construct
caaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgagcccgaacccttctgctttctattactagagcggccgctggccgcaataaaatatctttattttcattacatctgtgtgttgg

SEQ ID NO: 20 - DNA, 21 bp, Artificial Sequence
  Description of Artificial Sequence note = synthetic construct
cccuucugcuuucuauuactt

SEQ ID NO: 21 - DNA, 21 bp, Artificial Sequence
  Description of Artificial Sequence note = synthetic construct
ttgggaagacgaaagauaaug

SEQ ID NO: 22 - DNA, 21 bp, Artificial Sequence
  Description of Artificial Sequence note = synthetic construct
cccuucuuugcuacuuuactt

SEQ ID NO: 23 - DNA, 21 bp, Artificial Sequence
  Description of Artificial Sequence note = synthetic construct
ttgggaagaaacgaugaaaug

SEQ ID NO: 24 - DNA, 21 bp, Artificial Sequence
  Description of Artificial Sequence note = synthetic construct
ttgggaagacgaaagauaaug

SEQ ID NO: 25 - DNA, 21 bp, Artificial Sequence
  Description of Artificial Sequence note = synthetic construct
ttgggaagacgaaagauaaug

SEQ ID NO: 26 - DNA, 21 bp, Artificial Sequence
  Description of Artificial Sequence note = synthetic construct
ttgggaagacgaaagauaaug

SEQ ID NO: 27 - DNA, 21 bp, Artificial Sequence
  Description of Artificial Sequence note = synthetic construct
ttgggaagacgaaagauaaug

SEQ ID NO: 28 - DNA, 21 bp, Artificial Sequence
  Description of Artificial Sequence note = synthetic construct
ttgggaagacgaaagauaaug

SEQ ID NO: 29 - DNA, 21 bp, Artificial Sequence
  Description of Artificial Sequence note = synthetic construct
ttgggaagacgaaagauaaug

SEQ ID NO: 30 - DNA, 21 bp, Artificial Sequence
  Description of Artificial Sequence note = synthetic construct
ttgggaagacgaaagauaaug

SEQ ID NO: 31 - DNA, 21 bp, Artificial Sequence
  Description of Artificial Sequence note = synthetic construct
ttgggaagacgaaagauaaug

SEQ ID NO: 32 - DNA, 21 bp, Artificial Sequence
  Description of Artificial Sequence note = synthetic construct
ttgggaagacgaaagauaaug

SEQ ID NO: 33 - DNA, 23 bp, Artificial Sequence
  Description of Artificial Sequence note = synthetic construct
ggaaaggcaagagaaacggdtdt

SEQ ID NO: 34 - DNA, 23 bp, Artificial Sequence
  Description of Artificial Sequence note = synthetic construct
dtdtccuuuacguucucuuugac

SEQ ID NO: 35 - DNA, 23 bp, Artificial Sequence
  Description of Artificial Sequence note = synthetic construct
ggaaaugcaagagaaacugdtdt

SEQ ID NO: 36 - DNA, 23 bp, Artificial Sequence
  Description of Artificial Sequence note = synthetic construct
dtdtccuuuacguucucuuugac